(19) United States
(12) Patent Application Publication
(10) Pub. No.: US 2026/0086095 A1
(43) Pub. Date: Mar. 26, 2026

(54) METHODS FOR DETECTION OF PROSTATE CANCER, RELATED BIOMARKERS AND KITS FOR DETERMINING THE BIOMARKERS

(71) Applicant: OY ARCTIC PARTNERS AB, Turku (FI)

(72) Inventors: Kamlesh GIDWANI, Turku (FI); Henna KEKKI, Turku (FI); Kim PETTERSSON, Turku (FI)

(21) Appl. No.: 19/107,620

(22) PCT Filed: Aug. 31, 2023
(86) PCT No.: PCT/FI2023/050496

§ 371 Date: Feb. 28, 2025

(30) Foreign Application Priority Data
Sep. 1, 2022 (FI) 20225763

(51) Int. Cl. G01N 33/57555

(52) U.S. Cl. CPC G01N 33/57555; G01N 2333/4724; G01N 2333/96433

(57) ABSTRACT

The invention relates to non-invasive detection of prostate cancer on the basis of altered glycosylation patterns, and to biomarkers for use in the detection and to kits including reagents for determining levels of the biomarkers in biological samples.

### TECHNICAL FIELD

The present disclosure relates to non-invasive detection of prostate cancer on the basis of altered glycosylation patterns, especially of CA19-9-bearing entities and/or of free prostate specific antigen, and to said glycosylation patterns as biomarkers for use in the detection and to kits comprising reagents for determining levels of the biomarkers in biological samples.

### BACKGROUND

Prostate cancer is the second most common cancer in men worldwide. An estimated 1.4 million men were diagnosed with prostate cancer in 2020, accounting for 14.1% of the cancers diagnosed in men, with an estimated 375,304 deaths in 2020, prostate cancer is the fifth leading cause of male cancer deaths (6.8% of the total men deaths) worldwide.

Early detection and treatment can significantly improve prostate cancer survival. Prostate specific antigen (PSA) is the most frequently used biomarker to screen for prostate cancer. It is effective in detecting early-stage prostate cancers, but may sometimes miss harmful cancers. However, PSA is widely increased also in benign conditions and non-aggressive cancers and has, therefore, led to severe overdiagnosis and overtreatment of prostate cancer.

Modified assays detecting alternative molecular forms of PSA, including intact PSA (iPSA), free PSA (fPSA) have been developed in order to increase the diagnostic sensitivity and accuracy of total PSA for the early prostate cancer detection. These three PSA-based biomarkers along with a fourth prostatic biomarker, human kallikrein-related peptidase 2 (hK2), are included in OPKO Health's in vitro test, 4Kscore Test, recently approved by the US Food and Drug Administration (FDA) for identifying the risk of aggressive prostate cancer. The enhanced performance of this multi-kallikrein test over conventional PSA has been widely reported

Despite developments in the diagnostics of prostate cancer, there is still an identified need for biomarkers that can differentiate between prostate cancers with lower clinical significance from those prostate cancers that are likely to progress to an advanced or lethal disease if left untreated and that cause most of the morbidity and mortality related to the disease. Such markers would be highly beneficial in avoiding overtreatment of the less aggressive and slowly growing forms of prostate cancer, and in specifically targeting more radical forms of therapy to the clinically relevant forms of prostate cancer.

Glycosylation is one of the most significant post-translational protein modifications. It has been shown that glycosylation patterns of many proteins change during cancer initiation and progression. Indeed, some serum glycan structures have long been recognized as biomarkers in numerous types of cancers.

CA19-9 (sialylated Lex a) is one of several blood group carbohydrates identified by monoclonal antibodies that have found major applications as markers of cancer preferentially pancreatic cancer. It is a glycan, sialylated lacto-N-fucopenteose II ganglioside, and is a sialylated derivative of the Leₐ blood group antigen denoted as Leₓₐ. The expression of the CA19-9 antigen requires the Lewis gene product, 1,4-fucosyl transferase. CA19-9 is synthesized by normal human pancreatic and biliary ductular cells and by gastric, colon, endometrial, and salivary epithelia, In the circulation, it is reported to be present on several high molecular weight glycoprotein complexes and possibly other proteins. Patients who are genotypically Le_{a-b-} (about 5-10% of Caucasians) do not express CA19-9. The monoclonal antibody against CA19-9 was developed from a human colon carcinoma cell line, SW-1116. Several diagnostic companies have produced CA19-9 immunoassays. Considerable differences among assays are noted, depending on differences in the antibodies used and therefore assay results are not interchangeable for individual patients using different assay manufacturers. Typically, the CA19-9 reactive antibodies are used both as the capture and the detection antibody, i.e. based on a sandwich assay configuration. The established CA19-9 upper reference limit is 37 kU/L, as determined from the 99th percentile of normal subjects. This cut-off discriminates between pancreatic cancer and benign pancreatic disease with clinical sensitivities of 69 to 93% and clinical specificities of 76 to 99%. Elevated CA19-9 concentrations (>37 kU/L) can also be found in patients with hepatobiliary (67%), gastric (40 to 50%), hepatocellular (30 to 50%), colorectal (30%), and breast (15%) cancers. CA19-9 has no diagnostic or prognostic utility in prostate cancer detection and management

### SUMMARY

In one aspect, provided herein is a biomarker for prostate cancer, especially for clinically relevant prostate cancer. Said biomarker is a glycan structure that is comprised on a carbohydrate antigen 19-9 (CA19-9) bearing molecular entity and that is capable of specific binding to Mannose binding lectin (MBL). In another aspect, said biomarker is a glycovariant of free prostate specific antigen (fPSA) capable of specific binding to Macrophage galactose-type lectin (MGL).

In a further aspect, provided is a method of determining prostate cancer disease state in a subject. The method comprises: a) assaying a sample obtained from said subject for the level of a CA19-9-bearing entity comprising a glycan structure capable of specific binding to MBL; b) comparing the assayed level obtained in step a) with that of a control sample or a predetermined threshold value; and c) determining the prostate cancer disease state on the basis of said comparison. Alternatively or in addition, step a) may comprise assaying a sample obtained from said subject for the level of a glycovariant of fPSA capable of specific binding to MGL. Accordingly, step b) then comprises comparing the assayed level of said glycovariant of fPSA with that of a control sample or a predetermined threshold value; and c) determining the prostate cancer disease state on the basis of said comparison.

In a further aspect, provided is a kit for use in determining prostate cancer disease state in a subject. The kit comprises: i) a CA19-9-binding agent and ii) a binder molecule specific for a glycan structure capable of specific binding to MBL. Either said CA19-9-binding agent or said binder molecule comprises a detectable label. Alternatively or in addition, the kit comprises: i) a multi-well plate having a fPSA-binding agent immobilized on its bottom surface at an area having a size smaller than the size of the bottom surface, and ii) a binder molecule specific for a glycan structure capable of specific binding to MGL. Use of the kits in determining prostate cancer disease state in a subject is also provided.

In a still further aspect, provided is use of a binder molecule selected from the group consisting of binder molecules specific for a glycan structure capable of specific binding to MBL, binder molecules specific for a glycan structure capable of specific binding to WGA and binder molecules specific for a glycan structure capable of specific binding to MGL for determining the presence or absence of a prostate cancer-associated CA19-9-bearing entity in a sample.

Also provided is use of MBL or a binder molecule specific for a glycan structure capable of specific binding to MBL for determining prostate cancer disease state in a subject.

Further aspects, embodiments and details are set forth in following figures, detailed description, examples, and dependent claims.

### BRIEF DESCRIPTION OF THE DRAWINGS

The accompanying drawings illustrate several embodiments of the disclosed subject matter, and together with the description, serve to explain principles of the disclosed compositions and methods.

FIGS. 1A-1D show calibration curves for a conventional CA19-9 Elisa immunoassay (EIA), and CA19-9^{MGL}, CA19-9^{WGA} and CA19-9^{MBL} glycovariant assays of the invention, respectively.

FIGS. 2A and 2B show Box plot presentations for a conventional CA19-9 ELISA immunoassay and both median and mean values of CA19-9 concentrations (U/mL) for clinically relevant prostate cancer (PCa) and indolent disease, as well as the same for individual patient groups, respectively. No discrimination of clinically relevant PCa (Gleason ≥7) from indolent disease (benign cases and Gleason 6 grade cancers) nor separation of the different cancer groups from benign samples was achieved.

FIGS. 3A and 3B show Box plots for a CA19-9^{MBL} Glycovariant assay and both median and mean values of CA19-9^{MBL} (U/mL) for clinically relevant PCa (Gleason ≥7) and indolent disease (benign cases and Gleason 6 grade cancers), as well as the same for individual patient groups, respectively.

FIGS. 4A and 4B show Box plots for a CA19-9^{WGA} Glycovariant assay and both median and mean values of CA19-9^{WGA} (U/mL) for clinically relevant PCa and indolent disease as well as the same for individual patient groups, respectively.

FIGS. 5A and 5B show Box plots for a CA19-9^{MGL} Glycovariant assay and both median and mean values of CA19-9^{MGL} (U/mL) for clinically relevant PCa and indolent disease as well as the same for individual patient groups, respectively.

FIG. 6 shows receiver operating characteristic curves for conventional Elisa assay and the three glycovariant CA19-9 assays. All the CA19-9 glycovariants determined (CA19-9^{WGA}, CA19-9^{MBL} and CA19-9^{MGL}) significantly improved the discrimination of patients having a more aggressive prostate cancer (Gleason sum 7 to 10) compared with patients with a benign condition or Gleason 6 graded non-significant cancer when compared to the reference Elisa.

FIG. 7 shows receiver operating characteristic curves and the area under curve (AUC) values for a multi-kallikrein model (total PSA, free PSA, intact PSA and hK2), three CA19-9 glycovariants (CA19-9^{MBL}, CA19-9^{WGA} and CA19-9^{MGL}), the three CA19-9 glycovariants in combination with the multi-kallikrein markers or free PSA^{MGL} glycovariant. GV=glycovariant.

FIGS. 8A-8D show the conventional CA19-9 EIA, CA19-9^{MBL}, total PSA and the free PSA^{MGL} glycovariant concentrations among the CA19-9 negative (n=20) and CA19-9 positive (n=229) patients. Higher elevations in the total PSA as well as the free PSA^{MGL} glycovariant concentrations was observed in CA19-9 negative patients with aggressive PCa compared to CA19-9 positive patients.

FIGS. 9A and 9B show Box plots for the CA19-9^{DC-SIGN} Glycovariant assay for clinically relevant PCa and indolent disease, as well as the individual patient groups, respectively. NS=no statistical significance.

FIG. 10A shows dose response curves (mean fluorescence signals measured from triplicates) from LNCaP PSA used as the standard material in fPSA^{MGL} assay carried out either in a spot format or in whole well format.

FIG. 10B shows dose response curves for healthy seminal plasma PSA (□) and cancerous LNCaP PSA (⋄) obtained with the PSA^{MGL} glycovariant assay in the spot format; whereas FIG. 10C shows the same obtained with a conventional free PSA immunoassay.

FIGS. 11A and 11B show Box plots for the conventional free PSA immunoassay and for the present free PSA^{MGL} glycovariant assay, respectively. As shown in FIG. 11B, free PSA^{MGL} discriminates both Gle 7 and Gle 8-10 cancers from the combined group of benign+Gle 6 cancers with high statistically significance, whereas FIG. 11B demonstrates that no discrimination can be seen for the conventional free PSA. The free PSA^{MGL} concentration (ng/mL)* refers to that obtained using purified PSA from LNCaP cell culture for calibration. The relative content of the free PSA^{MGL} glycovariant in the LNCaP PSA preparation is not known.

FIG. 12 shows a ROC plot displaying AUC of free PSA^{MGL} glycovariant (black solid line) and free PSA (black dotted line) from Gle ≥7 (n=107) and benign+Gle 6 (n=142) PCa patients.

FIG. 13 shows a ROC plot displaying AUC of free PSA^{MGL} glycovariant (black solid line), free PSA^{AAL} glycovariant (black dotted line) as well as both used in combination (grey dotted line) from Gle ≥7 (n=107) and benign+Gle 6 (n=142) PCa patients.

### DEFINITIONS

Unless otherwise defined, the terms and expressions used in this specification and claims have the meanings generally applicable in the field of cancer diagnostics. Some of terms and expression used herein have the meanings as defined below:

As used herein, the meaning of a singular noun includes that of a plural noun and thus a singular term, unless otherwise specified, may also carry the meaning of its plural form. In other words, the term “a” or “an” may mean one or more.

As used herein, the term “or” in the claims is used to mean “and/or” unless explicitly indicated to refer to alternatives only or when the alternatives are mutually exclusive, although the disclosure supports a definition that refers to only alternatives and “and/or.”

As used herein, the terms “biological sample” and “sample” are interchangeable and refer in particular to a sample of a bodily fluid, such as ascites fluid, seminal plasma, urine, blood such as plasma or serum, and peritoneal cavity fluid, obtained from a subject. Generally, obtaining the sample to be analysed from a subject is not part of the present method for determining a subject's cancer disease state. A urine, blood, serum or plasma sample is the most preferred sample type to be used in the present method and all its embodiments. In some embodiments, the sample is an EDTA plasma sample.

In embodiments which concern assessment of the level of more than one biomarker, same or different samples obtained from a subject whose cancer disease state is to be determined may be used for each assessment. Said different samples may be of the same or different type.

As used herein, the terms “biomarker” and “marker” are interchangeable, and refer broadly to a molecular entity which is differentially present in a sample taken from a subject with cancer, or a certain grade of the cancer, as compared to a comparable sample take from a control subject, such as an apparently healthy subject or a subject with a different grade of the cancer.

As used herein, the terms “glycoform” and “glycovariant” are interchangeable and refer to a particular form of a glycosylated biomarker. That is, when the same molecular backbone that is part of a biomarker has the potential to be linked to different glycans or sets of glycans, then each different version of the biomarker is referred to as a “glycoform” or a “glycovariant”.

As used herein, the term “CA19-9” refers to Carbohydrate antigen 19-9, also known as Cancer antigen CA19-9. It is a sialylated derivative of the Lewis^{a} blood group antigen (sialyl-Lewis A; sLeA) found in many secreted glycolipids and glycoproteins, such as mucins and carcinoembryonic antigen (CEA). CA19-9 is a tumor marker predominantly associated with gastrointestinal malignancies including pancreatic, gall bladder, gastric, and colorectal cancers. Notably, CA19-9 cannot detect subjects who are sialyl Lewis^{a} blood group-negative, who account for ˜5% of the general population. As used herein, the terms “CA19-9” and “CA19.9” are interchangeable.

CA19-9 belongs to a glycan family of Lewis antigens. A common feature is the core N-acetyl-lactosamine (LacNAc), which is a disaccharide of galactose linked to N-acetylglucosamine. The monosaccharides fucose and sialic acid can be attached to the LacNAc in various linkages. A sulfate group also can be attached to the Galactose or N-Acetylglucosamine.

As used herein, the term “CA19-9^{MBL}” refers to a glycovariant of a CA19-9-bearing molecular entity capable of specific binding to MBL without limitation to any detection technique. The terms “CA19-9^{MBL}” and “CA19-9-bearing entity comprising a glycan structure capable of specific binding to MBL” are interchangeable with the term “MBL-binding glycovariant of a CA19-9-bearing entity”.

Likewise, the terms “CA19-9^{WGA}” and “CA19-9^{MGL}” refer to glycovariants of CA19-9-bearing entities capable of specific binding to WGA and MGL, respectively, without limitation to any detection technique. Also these terms are interchangeable with the terms “CA19-9-bearing entity comprising a glycan structure capable of specific binding to WGA”, “WGA-binding glycovariant of a CA19-9-bearing entity”, “CA19-9-bearing entity comprising a glycan structure capable of specific binding to MGL” and “MGL-binding glycovariant of a CA19-9-bearing entity”, respectively.

As used herein, the term “multi-kallikrein” refers to a panel of four prostatic kallikrein protein biomarkers, namely total, free and intact prostate-specific antigen (PSA) and kallikrein-related peptidase 2 (hK2).

As used herein, the term “fPSA^{MGL}” refers to a glycovariant of free PSA capable of specific binding to MGL without limitation to any detection technique. The term is interchangeable with the terms “MGL-binding glycovariant of free PSA”.

As used herein, the term “binder molecule” refers broadly to any molecule that is able bind a biomarker or a glycan part thereof. Non-limiting examples of binder molecules include lectins, antibodies, antibody mimetics, and oligonucleotide and peptide aptamers.

As used herein, the terms “CA19-9 binder” and “CA19-9-binding agent” refer to binder molecules specific for CA19-9. Preferably, such binder molecules are anti-CA19-9 antibodies, preferably monoclonal anti-CA19-9 antibodies.

As used herein, the terms “fPSA binder” and “fPSA-binding agent” refer to a binder molecule specific for free PSA. Preferably, such a binder molecule is an anti-fPSA antibody, preferably a monoclonal anti-fPSA antibody or an antigen-binding fragment (e.g. a Fab fragment, a F(ab)₂ fragment or a F(ab′)₂ fragment) or a single chain variant thereof. Preferably, said antigen-binding fragment is site-specifically biotinylated.

As used herein, the term “glycan binder” refers to a binder molecule that specifically binds to a certain glycan structure comprised on a molecular entity. Preferably, such a binder molecule is a lectin, especially a lectin selected from the group consisting of MBL, WGA and MGL, or an anti-glycan antibody, especially an anti-glycan antibody specific for glycan structures capable of being recognized by MBL, WGA or MGL. Notably, the term “glycan binder” does not encompass CA19-9 binders although they, too, specifically bind glycan structures since CA19-9 is a glycan. In other words, “CA19-9 binders” and “glycan binders”, as used herein, refer to different binder molecules, the latter referring to binder molecules specific for glycan structures other than CA19-9 comprised on CA19-9-bearing molecular entities.

Lectins are a group of carbohydrate binding proteins present in many plants, especially seeds, and in fungi, bacteria and animals.

As used herein, the term “antibody” refers generally to an immunoglobulin structure comprising two heavy chains and two light chains interconnected by disulfide bonds. Antibodies can exist as intact immunoglobulins or as any of a number of well-characterized antigen-binding fragments or single chain variants thereof, all of which are herein encompassed by the term “antibody”. Non-limiting examples of said antigen-binding fragments include Fab fragments, Fab′ fragments, F(ab)₂ fragments, F(ab′)₂ fragments, and Fv fragments. Said fragments and variants may be produced by recombinant DNA techniques, or by enzymatic or chemical separation of intact immunoglobulins as is well known in the art. Accordingly, the term “antigen-binding fragment” refers to any fragment of a monoclonal antibody regardless of its preparation technique, including enzyme used (e.g. pepsin, papain, bromelain), provided that the fragment has retained the antigen-binding specificity of the monoclonal antibody from which it derives.

As used herein, the term “subject” refers to an animal, preferably to a mammal, more preferably to a human, and most preferably to a male. Depending on an embodiment in question, said subject may suffer from cancer with or without diagnosis, be suspected to suffer from cancer, be at risk of cancer, or may have already been treated for cancer. Herein, the terms “human subject”, “patient” and “individual” are interchangeable.

As used herein, the term “benign condition”, and the like, refers to non-cancerous urological conditions including but not limited to benign prostatic hyperplasia, a common noncancerous enlargement of the prostate gland in aging men. Generally, BPH is considered neither as a premalignant lesion nor a precursor of carcinoma.

As used herein, the term “prostate cancer” refers to any cancer of the prostate gland. Prostate cancer typically grows slowly and initially remains confined to the prostate gland, where it may not cause serious clinical harm. While the slowly growing types of prostate cancer may need minimal or no treatment, some other types of prostate cancer are more aggressive and can spread to other body parts, i.e. metastasize. The Gleason scoring system is the most common prostate cancer grading system used. The scores are added together to come up with an overall score between 6 and 10. Gleason scores of 5 or lower are not used. Therefore, the lowest Gleason score is 6, which is a low-grade or clinically insignificant cancer. A Gleason score of 7 is a medium-grade cancer, whereas scores of 8-10 are high-grade cancers. Gleason scores 7-10 are herein referred to collectively as “clinically relevant prostate cancers”.

As used herein, the term “indolent disease” refers collectively to benign urological conditions and to clinically insignificant Gleason score 6 graded prostate cancers.

As used herein, the term “indicative of cancer”, when applied to a biomarker, refers to a level which, using routine statistical methods setting confidence levels at a minimum of 95%, is diagnostic of said cancer or a stage of said cancer such that the detected level is found significantly more often in subjects with said cancer or a stage of said cancer than in subjects without said cancer or another stage of said cancer.

As used herein, the term “level” is interchangeable with the terms “amount” and “concentration”, unless otherwise indicated.

To determine whether a detected level of a biomarker is indicative of the presence or risk of the presence of a cancer associated with said biomarker, its level in a relevant control has to be determined. Once the control levels are known, the determined marker levels can be compared therewith and the significance of the difference can be assessed using standard statistical methods. In some embodiments, a statistically significant difference between the determined biomarker level and the control level is indicative of clinically relevant prostate cancer. In some further embodiments, before to be compared with the control, the biomarker levels are normalized using standard methods.

Comparison of the assayed level of a biomarker in a sample to be analysed with that of a relevant control or a predetermined threshold value may in some embodiments be performed by a processor of a computing device.

Regardless of whether or not the processor of the computing device is used for said comparison, the level of the assayed level of a biomarker is, at least in some embodiments, determined as “increased” or “higher” if the level of the biomarker in the sample is, for instance, at least about 1.5 times, at least about 1.75 times, at least about 2 times, at least about 3 times, at least about 4 times, at least about 5 times, at least about 6 times, at least about 8 times, at least about 9 times, at least about 10 times, at least about 20 times or at least about 30 times the predetermined threshold level or the level of the biomarker in a control sample. In some embodiments, the difference between the level of the biomarker in the sample to be analyzed and the predetermined threshold level or the level of the biomarker in a control sample has to be statistically significant in order to provide a proper diagnostic, prognostic or predictive result.

Concentration of a biomarker in a sample obtained from a subject whose cancer disease state is to be determined or who is to be screened, diagnosed, prognosed, or monitored for cancer is considered “non-increased” or “normal” if the detected concentration thereof is lower, essentially the same or essentially non-altered as compared with that of a relevant control sample or a predetermined threshold value.

As used herein, the term “control” may refer to a control sample obtained from an apparently healthy individual or pool of apparently healthy individuals, or it may refer to a control sample obtained from an individual or a pool of individuals with a benign condition such as a benign urological condition such as benign prostatic hyperplasia, or it may refer to a predetermined threshold value, i.e. a cut-off value, which is indicative of the presence or absence of clinically relevant prostate cancer. For example, the predetermined threshold value may refer to a biomarker level above which the subject is likely to have clinically relevant prostate cancer and/or below which the subject is unlikely to have clinically relevant prostate cancer. Statistical methods for determining appropriate threshold values will be readily apparent to those of ordinary skill in the art. The threshold values may have been determined, if necessary, from samples of subjects of the same age, demographic features, and/or disease status, etc. The threshold value may originate from a single individual not affected by clinically relevant prostate cancer or be a value pooled from more than one such individual.

In some embodiments, the term “control sample” refers to a sample obtained from the same subject whose prostate cancer disease state is to be determined but obtained at a time point different from the time point of the disease state determination. Non-limiting examples of such different time points include one or more time points before diagnosis of the disease, one or more time points after diagnosis of the disease, one or more time points before treatment of the disease, one or more time points during treatment of the disease, and one or more time points after treatment of the disease. Typically, such control samples obtained from the same subject are used when the purpose of cancer disease state determination is to monitor said disease, especially to monitor the onset of the disease, or risk development of the disease, response to treatment, relapse of the disease, or recurrence of the disease.

As used herein, the term “cancer disease state” refers to any distinguishable manifestation of cancer, including non-cancer. For example, the term includes, without limitation, information regarding the presence or absence of cancer, the presence or absence of a preclinical phase of cancer, the risk of having or developing cancer, the stage or grade of cancer, and progression of cancer.

As used herein, the term “spot assay” refers to an assay format wherein a binder molecule, more specifically a capturing agent, is immobilized on a solid surface at an area (i.e. a spot) that is smaller than the residual surface. In other words, in case of a multi-well assay plate, such as a microtiter plate, the spot has a size, more specifically a diameter, that is smaller than the size (diameter) of the well carrying the spot. Assay formats in which a single well contains multiple spots, each immobilized with a different binder molecule, are denoted herein as “array-in-well” assays. Assay formats in which the area immobilized with the binder molecule covers at least the whole bottom surface are denoted herein as “whole well assays”. Immobilization techniques for each of the assay formats are readily available in the art.

Some further terms and expressions used in this disclosure are explained below in the detailed description of the invention.

### DETAILED DESCRIPTION

### Biomarkers and Clinical Methods

The present disclosure is based on studies aiming to identify cancer-related glycovariants of molecular entities with improved sensitivity over other variants of the same entities as cancer biomarkers. In accordance with this aim, the present disclosure provides means and methods for determining prostate cancer disease state in a subject who is suspected to suffer from or being at risk of suffering from prostate cancer. Said means and methods are provided especially for screening, diagnosing, prognosing or monitoring prostate cancer.

It has now been surprisingly revealed that some glycovariants of CA19-9-bearing molecular entities can serve as biomarkers of prostate cancer. Earlier, CA19-9 has been predominantly associated with gastrointestinal malignancies such as pancreatic, gall bladder, gastric, and colorectal cancers.

Conventional CA19-9 immunoassays are based on the recognition of the CA19-9 antigen in a sample such as serum or plasma by using either the same (monoclonal) antibody or alternatively two different (monoclonal) antibodies both recognizing, the CA19-9 antigen. The actual glycoprotein carrying the CA19-9 antigens and thus determined with the CA19-9 assay has not been exactly characterized but is likely to be any of several proteins of the mucin family. However also other proteins may be carriers of the CA19-9 antigen.

It is shown herein that detection of certain glycovariants of CA19-9-bearing molecular entities instead of using conventional CA19-9 immunoassays enables distinguishing subjects with indolent urological disease from subjects with clinically relevant prostate cancer among subjects suspected of having prostate cancer.

In the experiments leading to some embodiments of the present invention, Mannose binding lectin (MBL) showed excellent discrimination (p=0.000003) between CA19-9-bearing entities derived from subjects with a benign urological condition or Gleason 6 prostate cancer and CA19-9-bearing entities derived from subjects with clinically relevant prostate cancer. As known in the art, MBL has predominant affinity to fucose and mannose/mannan. Interestingly, another lectin having affinity to high mannose-type structures, namely Dendritic Cell-Specific Intercellular adhesion molecule-3-Grabbing Non-integrin (DC-SIGN), showed no corresponding discrimination between different patient groups among subjects suspected of having prostate cancer.

In accordance with the above, by assaying biological samples obtained from subjects suspected of having prostate cancer for CA19-9-bearing molecular entities capable of specific binding to MBL, subjects with indolent urological disease can be distinguished from subjects with clinically relevant prostate cancer. No such distinction is possible by conventional CA19-9 immunoassays.

Therefore, in one aspect, provided herein is an MBL-binding glycovariant of a CA19-9-bearing entity as a biomarker for prostate cancer, especially clinically relevant prostate cancer. Notably, this prostate cancer biomarker of the invention is independent from the prostate gland volume, as is demonstrated in Example 4 below. In other aspects, provided are use of the MBL-binding glycovariant of a CA19-9-bearing entity in detecting clinically relevant prostate cancer and various clinical methods discussed in more detail below that employ the biomarker.

Clear discrimination between subjects with indolent disease and subjects with clinically relevant prostate cancer was seen also in experiments with Wheat germ agglutinin (WGA) and Macrophage galactose-type lectin (MGL). To be more specific, among lectins tested, also WGA and MGL showed excellent discrimination between CA19-9-bearing entities derived from subjects with a benign urological condition or Gleason 6 prostate cancer and CA19-9-bearing entities derived from subjects with clinically relevant prostate cancer.

Accordingly, provided herein are WGA-binding as well as MGL-binding glycovariants of CA19-9-bearing entities as independent biomarkers for prostate cancer, especially clinically relevant prostate cancer. Also provided are uses of these biomarkers in detecting clinically relevant prostate cancer and various clinical methods that employ these biomarkers.

Biomarkers of the invention may be used in different combinations. While some embodiments may concern any of the three above-disclosed biomarkers alone, some other embodiments may concern any two of the three biomarkers, i.e., a combination of MBL-binding and WGA-binding glycovariants of CA19-9-bearing entities, a combination of MBL-binding and MGL-binding glycovariants of CA19-9-bearing entities, or a combination of WGA-binding and MGL-binding glycovariants of CA19-9-bearing entities. Some further embodiments may concern all three biomarkers. Notably, as demonstrated in the examples, WGA-binding glycovariants of CA19-9-bearing entities as well as MGL-binding glycovariants of CA19-9-bearing entities complemented the diagnostic performance of MBL-binding glycovariants of CA19-9-bearing entities.

In some embodiments, the biomarkers of the invention may be used in combination with one or more known biomarkers of prostate cancer, such as total PSA, free PSA, intact PSA, hK2 and/or fPSA^{MGL}. Notably, as demonstrated in the examples, each of CA19-9^{MBL}, CA19-9^{WGA} and CA19-9^{MGL} biomarkers outperformed free PSA, intact PSA, total hK2 and free hK2 in distinguishing clinically relevant cancers from indolent diseases. Also the combination of CA19-9^{MBL}, CA19-9^{WGA} and CA19-9^{MGL} outperformed the multi-kallikrein panel of biomarkers in this aim. Furthermore, the performance of the CA19-9 glycovariants was improved when used in combination with the multi-kallikrein panel.

Interestingly, CA19-9 negativity appeared to indicate more aggressive prostate cancer as judged by a shift to higher-grade prostate cancers, as well as to higher total PSA and fPSA^{MGL} levels in the study cohort. CA19-9 negative subjects accounted for 8% of the cohort, which number is in accordance with the general understanding that 5-10% of Caucasian individuals are Lewis antigen negative and hence miss CA19-9. Accordingly, in one aspect, the present invention provides CA19-9 negativity as a novel marker for more aggressive prostate cancer in subjects already diagnosed with prostate cancer.

Notably, despite being negative for CA19-9 as determined by a conventional CA19-9 immunoassay, CA19-9^{MBL} could still distinguish subjects with indolent disease from subjects with clinically relevant prostate cancer in a cohort of study subjects classified as CA19-9 negative.

In an aspect of the present disclosure, fPSA^{MGL} is provided as a blood-based biomarker of prostate cancer, especially clinically relevant prostate cancer, with or without concomitant use of herein-disclosed glycovariants of CA19-9-bearing entities and/or known biomarkers of prostate cancer. Earlier, fPSA^{MGL} has been suggested as a biomarker of prostate cancer (WO2018011474) but, while performing well with urine samples and cancerous prostate tissue lysates, it has not performed well with blood samples. Unexpectedly, it has now been realized that fPSA^{MGL} performs well also with blood samples if a spot assay format is utilized. Indeed, it is shown herein that detection of fPSA^{MGL} in blood samples using said assay format enables distinguishing subjects with indolent urological disease from subjects with clinically relevant prostate cancer among subjects suspected of having prostate cancer. Notably, fPSA^{MGL} does not show gland volume dependency, performing equally in different gland volume groups.

As already indicated above, the biomarkers of the invention, or combinations thereof, may be employed in various prostate cancer-related clinical methods with or without additional use of known biomarkers of prostate cancers. Such methods may be defined or expressed in different ways. For example, in some embodiments the method is a method of detecting prostate cancer, especially a clinically relevant prostate cancer, or a method of determining prostate cancer disease state in a subject. The method may also be expressed as a method of identifying a subject as having clinically relevant prostate cancer or being at risk of having or developing clinically relevant prostate cancer. Moreover, the method may also be expressed as a method of diagnosing prostate cancer, i.e., determining whether or not a subject has or is at risk of having or developing prostate cancer, especially clinically relevant prostate cancer. Further ways of expressing the methods are apparent to those skilled in the art. Usually, the subject whose prostate cancer disease state is to be determined is suspected of having prostate cancer. In essence, the method steps are the same regardless of how the purpose of the method is expressed.

The above-discussed clinical methods are also meant to include instances where the presence or the risk of prostate cancer is not finally determined but that further diagnostic testing is warranted. In such embodiments, the method is not by itself determinative of the presence or absence, or of the risk of prostate cancer in the subject but can indicate that further diagnostic testing is needed or would be beneficial. Therefore, the present methods may be combined with one or more other diagnostic methods for the final determination of the presence or absence, or of the risk of prostate cancer in the subject. Such other diagnostic methods are well known to a person skilled in the art.

Being non-invasive and suitable for analyzing samples of bodily fluids, the present clinical methods and their various embodiments may be easily incorporated into a population screening protocol to identify subjects having or being at risk of having or developing prostate cancer. This would enable not only early diagnosis, but also active surveillance for the onset of clinically relevant prostate cancer in subjects with identified increased risk of developing prostate cancer later in life. Moreover, early detection of clinically relevant cancer would allow treating the disease early when chances of cure are at their highest.

It is envisaged that the present methods and their various embodiments may be used not only for diagnostic purposes but also for different monitoring purposes. Such monitoring purposes include without limitation monitoring onset of clinically relevant prostate cancer in a subject suspected of having or being at risk of prostate cancer, and monitoring for any development in a subject's prostate cancer disease state including monitoring for the subject's recovery or survival from cancer, any possible relapse or recurrence of the disease, or response to treatment. In such embodiments, the method comprises monitoring prostate cancer in said subject by comparing the level of one or more biomarkers of the invention (more specifically, one or more CA19-9-bearing entities comprising glycan structures capable of specific binding to MBL, WGA and/or MGL; and/or fPSA^{MGL}) with the respective level in one or more other samples obtained from the same subject at a different time point. Samples that may be employed in the monitoring include, but are not limited to, samples collected at different time points after diagnosis of cancer and/or before, during, and after therapeutic intervention, e.g. by surgery, radiation therapy, chemotherapy, any other suitable therapeutic treatment, or any combination thereof, to relieve or cure cancer. In some embodiments, said monitoring is carried out by repeating the assaying step at least twice at different time points, wherein said time points are selected, independently from each other, from the time points set forth above. In some embodiments, the monitoring is carried out during or after treatment of cancer, and/or the method comprises determining said subject as having relapse or recurrence of cancer or as being at risk of relapse or recurrence of cancer, if the level of at least one of the biomarkers is higher than in one or more earlier samples obtained from the same subject, or higher than in a relevant control or above a predetermined threshold value.

In some implementations, the present methods may further include therapeutic intervention. Once a subject is determined to have or be at risk of having or developing clinically relevant prostate cancer, he may be subjected to cancer treatment that is predicted to be effective. Such methods may be formulated in different ways. For example, in some embodiments, the present invention provides a method of determining a subject as having clinically relevant prostate cancer or being at risk of having or developing cancer, the method comprising a) assaying a sample obtained from the subject for the level of a CA19-9-bearing entity comprising a glycan structure capable of specific binding to a given lectin, b) comparing the determined level of said entity with the level of said entity in a control sample or with a predetermined threshold value and c) responsive to the comparison, determining the presence of clinically relevant prostate cancer or the risk of such cancer in the subject, wherein increased level of said entity in the sample obtained the subject as compared to the level of a corresponding entity in the control or to the predetermined threshold value indicates that the subject has or is at risk of having or developing clinically relevant prostate cancer, and d) providing cancer treatment to the subject. Alternatively or in addition, step a) may comprise assaying a sample obtained from said subject for the level of a glycovariant of fPSA capable of specific binding to MGL. In such instances, step b) comprises comparing the assayed level of said glycovariant of fPSA with that of a control sample or a predetermined threshold value; the prostate cancer disease state is determined on the basis of said comparison in step c); and cancer treatment is administered to the subject in step d).

In the present context, the term “treatment” refers to providing cancer treatment to a subject in need thereof for purposes which may include ameliorating, lessening, inhibiting, or curing cancer. Treatment for cancer may involve one or more therapies selected from surgery, chemotherapy, radiation therapy, immunotherapy or targeted therapy such as therapy with small molecule inhibitors.

Furthermore, as developing new medical drugs and therapies for subjects suffering from prostate cancer is challenged by the highly variable cancer-related morbidity and mortality, markers allowing more accurate selection of subjects with clinically relevant forms of prostate cancer for therapeutic trials are crucially needed to facilitate the development of new or alternative treatment strategies especially for the aggressive forms of the disease. It is envisaged that the biomarkers of the invention are suitable for this purpose.

Details and embodiments of the present biomarkers and clinical methods disclosed above apply also to assays, assay formats and kits disclosed below as is readily understood by a skilled person, even if the details and embodiments are not repeated. Accordingly, details and embodiments disclosed below with respect to the present assays, assay formats and kits apply also to the biomarkers and clinical methods disclosed above the way appropriate and readily understood by a skilled person, even if the details and embodiments are not repeated.

### Assay Formats

As already indicated above, the biomarkers of the invention or combinations thereof may be employed in various prostate cancer-related clinical methods. Such methods may be defined or expressed in different ways. For example, some embodiments provide a method of determining prostate cancer disease state in a subject, in which method a sample obtained from said subject is first assayed for the level of CA19-9-bearing entity comprising a glycan structure capable of specific binding to a given lectin and/or for fPSA^{MGL}. In a second step, the assayed level of said entity or said fPSA^{MGL} is compared with that of a control sample or a predetermined threshold value. The subject's prostate cancer disease state is then determined on the basis of said comparison. In some embodiments increased level of said entity is indicative of clinically relevant prostate cancer. On the other hand, non-increased level of said entity as compared to the level of the entity in a control sample or a predetermined threshold value is indicative of a benign urological condition or clinically insignificant (Gleason 6) prostate cancer. In accordance with what is stated above, the lectin in question for the CA19-9-bearing entity is either MBL, WGA or MGL, preferably MBL. In some embodiments, the method may comprise assaying the sample for more than one type of CA19-9-bearing entities, each being capable of specific binding to a different lectin. In some preferred embodiments, the sample is assayed for CA19-9-bearing entities capable of specific binding to MBL and additionally either to WGA or MGL. In some further embodiments, the sample is assayed for CA19-9-bearing entities capable of specific binding to MBL, for CA19-9-bearing entities capable of specific binding to WGA and also for CA19-9-bearing entities capable of specific binding to MGL. Assaying for multiple glycovariants of CA19-9-bearing entities can be carried out either in a same assay (i.e. concomitantly) or in different assays (i.e. in parallel), either simultaneously or sequentially. In some embodiments, the method may comprise assaying the sample for fPSA^{MGL} with or without assaying the sample also for one or more CA19-9-bearing entities in any combination disclosed above. Again, if the method involves assaying for multiple biomarkers, they can be carried out either in a same assay or in different arrays, or in different assays, either simultaneously or sequentially, as desired. Carrying out in the same assay may be achieved by multiplexing or by using different wells or arrays in a multi-well plate, preferably a microtiter plate. In some embodiments, the assaying may be carried out in an array-in-well format, wherein a single well contains distinct spots immobilized with a CA19-9-binding agent and a fPSA-binding agent, preferably an antigen-binding fragment of an anti-fPSA antibody, such as a Fab, F(ab)₂ or F(ab′)₂ fragment, more preferably a site-specifically biotinylated antigen-binding fragment of an anti-fPSA antibody. In some other embodiments, the assaying may be carried out on a multi-well plate comprising one or more wells immobilized with a CA19-9-binding agent and one or more different wells immobilized with a fPSA-binding agent, preferably an antigen-binding fragment of an anti-fPSA antibody, such as a Fab, F(ab)₂ or F(ab′)₂ fragment, more preferably a site-specifically biotinylated antigen-binding fragment of an anti-fPSA antibody.

Before elaborating the step of assaying a sample for the level of a CA19-9-bearing entity comprising a glycan structure capable of specific binding to a given lectin, the following is to be noted. Extracellular vesicles (EVs) are lipid bilayer-covered particles that are naturally secreted from cells, and found in biological fluids including, but not limited to, blood, urine and cerebrospinal fluid. EVs carry cargos of proteins, nucleic acids, lipids and metabolites from the parent cell, and may present various antigens such as CA19-9 on their surfaces. However, CA19-9 may also be found in biological fluids as soluble glycoconjugates, without being attached to vesicles. Thus, the present methods encompass determining levels of certain glycan structures in biological samples regardless of whether they exist as glycovariants of soluble glycoconjugates or are comprised on EV's. Thus, assaying a sample for CA19-9-bearing entities refers not only to instances wherein the sample is assayed for the presence of CA19-9-bearing soluble glycoconjugates comprising a glycan structure capable of specific binding to a given lectin but also to instances wherein the sample is assayed for EVs presenting both CA19-9 and a glycan structure capable of binding to the given lectin on its surface. Such assaying may be achieved by assaying the sample for those CA19-9-bearing entities (soluble glycoconjugates or EVs) that can be captured by an anti-CA19-9 antibody and that comprise a glycan structure capable of specific binding to the given lectin However, some embodiments may specifically concern glycovariants of soluble glycoconjugates comprising CA19-9, while some other embodiments may specifically concern glycan structures presented by CA19-9-presenting EVs.

Assaying for the level of a CA19-9-bearing entity comprising a glycan structure capable of specific binding to a given lectin can be achieved by different means and methods readily available in the art. In some embodiments, glycan binders such as lectins or anti-glycan antibodies can be utilized. Such embodiments may comprise contacting a sample obtained from a subject suspected of having prostate cancer with a CA19-9 binder, such as an anti-CA19-9 antibody, and with a glycan binder specific for a glycan structure capable of specific binding to a given lectin, i.e., any one of MBL, WGA and MGL, and detecting a resulting binding reaction, the extent of which is indicative of the level of the CA19-9-bearing entity of interest in said sample. In some embodiments, the glycan binder is a lectin, more specifically MBL, WGA or MGL, or an antibody capable of specific binding to the same glycan structure as MBL, WGA, or MGL. A corresponding step may be applied to a control sample, to enable comparison of the level of the CA19-9-bearing entity of interest in a sample obtained from the subject suspected of having prostate cancer and the level of the same in a relevant control sample.

Accordingly, assaying for the level of a CA19-9 bearing entity comprising a glycan structure capable of specific binding to MBL (CA19-9^{MBL}), may be carried out by employing an anti-CA19-9 antibody and MBL. However, the term “capable of specific binding to MBL” is not limited to the use of MBL in the detection. In other words, CA19-9^{MBL} may be detected or assayed not only by techniques employing MBL but also by other techniques such as those employing lectins other than MBL having the capability of specifically recognizing MBL-binding species of CA19-9 owing to a similar or overlapping binding specificity with that of MBL. Specificities of different lectins and, thus, their suitability for this purpose can be easily determined by those skilled in the art. Also anti-glycan antibodies specific for the glycan structure specifically recognizable by MBL may be employed as binder molecules for detecting CA19-9^{MBL}

Likewise, assaying for the level of a CA19-9 bearing entity comprising a glycan structure capable of specific binding to WGA (CA19-9^{WGA}), may be carried out by employing an anti-CA19-9 antibody and WGA. However, the term “capable of specific binding to WGA” is not limited to the use of WGA in the detection. In other words, CA19-9^{WGA} may be detected or assayed not only by techniques employing WGA but also by other techniques such as those employing lectins other than WGA having the capability of specifically recognizing WGA-binding species of CA19-9 owing to a similar or overlapping binding specificity with that of WGA. Specificities of different lectins and, thus, their suitability for this purpose can be easily determined by those skilled in the art. Also anti-glycan antibodies specific for the glycan structure specifically recognizable by WGA may be employed as binder molecules for detecting CA19-9^{WGA}.

Likewise, assaying for the level of a CA19-9 bearing entity comprising a glycan structure capable of specific binding to MGL (CA19-9^{MGL}), may be carried out by employing an anti-CA19-9 antibody and MGL. However, the term “capable of specific binding to MGL” is not limited to the use of MGL in the detection. In other words, CA19-9^{MGL} may be detected or assayed not only by techniques employing MGL but also by other techniques such as those employing lectins other than MGL having the capability of specifically recognizing MGL-binding species of CA19-9 owing to a similar or overlapping binding specificity with that of MGL. Specificities of different lectins and, thus, their suitability for this purpose can be easily determined by those skilled in the art. Also anti-glycan antibodies specific for the glycan structure specifically recognizable by MGL may be employed as binder molecules for detecting CA19-9^{MGL}.

Assaying for the level of a CA19-9-bearing entity comprising a glycan structure capable of specific binding to a given lectin can be carried out, for example, by using a sandwich assay, wherein a CA19-9 binder molecule, such as an anti-CA19-9 antibody, preferably a monoclonal antibody, is used as a capturing agent and a glycan binder having a desired binding specificity, such as a lectin or a corresponding anti-glycan antibody, is used as a tracer. For use as a tracer, said glycan binder may be detectably labelled, either directly or indirectly.

In some other embodiments, a sandwich assay may be conducted using a reversed way. In such cases, a glycan binder, such as a lectin or a corresponding anti-glycan antibody, is used as a capturing agent and a CA19-9-specific binder molecule, such as an antibody, preferably a monoclonal antibody, is used as a directly or indirectly detectably labelled tracer. It is envisaged that since urine contains less interfering glycosylated molecules than blood, the reversed sandwich assay may operate better with urine samples than with blood samples.

Notably, sandwich assays may be employed regardless of whether the glycan structure whose level is to be assayed is on a soluble CA19-9 bearing glycoconjugate or on a CA19-9-presenting vesicle.

In some embodiments, a sandwich assay may comprise one or more washing steps after a capturing step in order to remove all non-targeted and unbound molecular entities not specific for the capturing agent. Appropriate washing solutions and conditions (e.g., time and temperature) are known to those skilled in the art. After the capturing step and, optionally, one or more washing steps, the captured molecular entities are subjected to a detection step by employing an appropriate tracer.

Sandwich assays according to various embodiments of the present invention may be performed either on a solid surface, such as on a microtiter plate, or in lateral flow format. Means and methods for binding a capturing agent to a solid surface, e.g., via a streptavidin-biotin complex, or incorporating a capturing agent to a lateral flow assay are known in the art and readily apparent to a skilled person.

Suitable substrates for use in the present solid phase sandwich assays include, but are not limited to, glass, silica, aluminosilicates, borosilicates, metal oxides such as alumina and nickel oxide, gold, various clays, nitrocellulose or nylon. As indicated above, the substrate may in some embodiments be coated with an appropriate compound, such as streptavidin to enhance binding of a capturing agent (i.e. either a CA19-9 binder or a glycan binder) to the substrate. In some further embodiments, one or more control binders, such as control antibodies or control lectins, may also be attached to the substrate.

In some embodiments, the solid phase sandwich assay may be provided in spot format, i.e. as an assay wherein the CA19-9 capturing agent, preferably an antibody, more preferably a monoclonal antibody or an antigen binding fragment or a single-chain variant thereof is immobilized as a spot on a multi-well plate, preferably of a microtiter plate, instead of the assay being a conventional whole well assay, wherein the capturing agent is immobilized over the whole surface area of a multi-well plate, preferably a microtiter plate. Typically, the diameter of the spot area is 1-5 mm, 1-3 mm, or 2.5-3 mm. Density of the capturing agent may vary, for example depending on the capturing agent in question. However, typically the concentration of the capturing agent to be immobilized varies from 25 to 150 μg/ml, preferably from 50 to 125 μg/ml or from 75 to 100 μg/ml.

In some embodiments, the solid phase sandwich assay may be provided in conventional whole well assay format.

Anti-CA19-9 antibodies are commercially available from different sources, and may be used in the above-mentioned sandwich assays either as capturing agents or as tracers. Further CA19-9-specific monoclonal antibodies may be produced according to methods well known in the art. For use as a tracer, an anti-CA19-9 antibody may be labelled with any appropriate label known in the art including, but not limited, to fluorescent labels, bioluminescent labels, chemiluminescent labels and enzyme labels such as alkaline phosphatase. Those skilled in the art can easily selected an appropriate detection technique depending on the type of a detectable label employed.

Lectins are commercially available from multiple sources. Also anti-glycan antibodies with desired specificities towards carbohydrate antigens are commercially available from multiple sources, or may be produced according to methods known in the art. For use as a tracer, a glycan binder, such as a lectin or a corresponding anti-glycan antibody, may be detectably labelled by methods well known in the art. In some embodiments, the glycan binder to be employed may be directly labelled with any available detectable label using standard techniques. In some other embodiments, the glycan binder to be employed may be detectably labelled indirectly, for example by immobilizing it on a detectable particle, such as a nanoparticle. Furthermore, in those embodiments which involve more than one different glycan binders, they may be labelled, either directly or indirectly, with the same or different labels. In some embodiments, multiplexing may be achieved by using differently labelled nanoparticles bearing different glycan binder species in a single assay.

As used herein, the term “nanoparticle” (NP) refers to a particle, synthetic or natural, having one or more dimensions, e.g. a diameter, of less than about 1000 nm, e.g. about 500 nm or less, about 100 nm or less, or about 50 nm or less. As used herein, the term “about” refers to a range of values±10% of a specified value. For example, the phrase “about 100 nm” includes ±10% of 100 nm, or from 90 nm to 110 nm. The nanoparticles may generally have a spherical shape but also non-spherical shapes such as ellipsoidal shapes can be used. In some embodiments, all the dimensions of said nanoparticle are less than about 1000 nm, about 500 nm or less, about 100 nm or less, or about 50 nm or less.

A variety of different materials may be utilized in the present nanoparticles. Non-limiting examples of suitable polymers include poly(ethylene glycol) (PEG), polystyrene, polyethylene, poly(acrylic acid), poly(methyl methacrylate) (PMMA), polysaccharides, and copolymers or combinations thereof. Other suitable nanoparticle materials include, but are not limited to, colloidal gold, silver, quantum dots, carbon, porous silicon, and liposomes. Further suitable nanoparticle materials include protein nanoparticles, mineral nanoparticles, glass nanoparticles, nanoparticle crystals, metal nanoparticles, and plastic nanoparticles.

Nanoparticles suitable for use in various embodiments of the present invention may be directly or indirectly qualitatively or quantitatively detectable by any known means. For instance, the nanoparticles may be detectable owing to an inherent quality as in the case of e.g. upconverting (UCP) nanoparticles, resonance particles, quantum dots, and gold particles. UCP particles are particularly suitable for use as tracers in lateral flow formats. In some other embodiments, the nanoparticles can be made detectable e.g. by fluorescent labels, bioluminescent labels, chemiluminescent labels. In some further embodiments, labelling or doping with lanthanides, i.e. luminescent lanthanide ions with luminescence emission in visible or near-infrared or infrared wavelengths and long fluorescence decay, such as europium (III), terbium (III), samarium (III), dysprosium (III), ytterbium (III), erbium (III) and neodynium (III), are preferred means for making the present nanoparticles detectable.

Glycan binders, such as lectins or anti-glycan antibodies, may be immobilized on nanoparticles by any suitable method known in the art, including but not limited to that disclosed in the examples herein. In those embodiments which involve more than one different glycan binders, including e.g. more than one different lectin and/or more than one different anti-glycan antibody, said different glycan binders may be immobilized either on the same or different nanoparticles in any desired ratios. Notably, said different glycan binders may be of different types (e.g. either lectins, anti-glycan antibodies, antibody mimetics or aptamers) or differ in their glycan specificities, or both.

In some non-limiting embodiments, the most preferred nanoparticles are polystyrene nanoparticles having a diameter of either about 97 nm or about 107 nm, for example. Such nanoparticles are commercially available at least from Thermo Scientific Seradyn Inc. Further preferred nanoparticles include europium chelate-doped nanoparticles.

Advantages associated with the use of nanoparticles include i) signal amplification provided by a great number of chelates per particle, ii) strengthened functional affinity (avidity) of the lectins or anti-glycan antibodies to their target glycostructure epitopes enabled by the high density of immobilized lectins or anti-glycan antibodies on the particle, and iii) the glycostructure specificity of the lectins or anti-glycan antibodies used as enabled by creation of the multivalent nanoparticles. However, nanoparticles are only one preferred way of providing adequate avidity effect and signal amplification for carrying out the present invention or disclosure and their various embodiments.

It is also possible to create a detectable signal by using any available sensor technology. For example, a solid surface may incorporate a recognition element (transducer) capable of converting the binding reaction into a detectable signal with or without the use of label moieties. Different types of transducers can be employed, including those based on electrochemical or optical detection. Detection may also be based on either homogeneous or heterogeneous detection techniques, as is apparent to those skilled in the art.

Moreover, assaying a sample for a CA19-9-bearing entity comprising a glycan structure capable of specific binding to a given lectin be carried out by methods or techniques such as, nuclear magnetic resonance (NMR), electrophoresis, chromatographic methods and mass spectrometry, or any appropriate combinations thereof. Suitable NMR methods include, for example, correlation spectroscopy (COSY), total correlation spectroscopy (TOCSY), nuclear overhauser effect spectroscopy (NOESY) and rotating frame overhauser enhancement spectroscopy (ROESY), all of which methods may be used in either 1D or 2D. Suitable electrophoretic methods include, for example, capillary electrophoresis with laser induced fluorescence (CE-LIF), capillary gel electrophoresis (CGE) and capillary zone electrophoresis (CZE). Non-limiting examples of mass spectrometric methods include fast atom bombardment mass spectrometry (FAB-MS), Fourier transform ion cyclotron resonance mass spectrometry (FTICR-MS), liquid chromatography mass spectrometry (LC-MS), liquid chromatography tandem mass spectrometry (LC-MS/MS), liquid chromatography with electrospray ionization mass spectrometry (LC-ESI-MS), matrix-assisted laser desorption/ionization mass spectrometry (MALDI-MS), matrix-assisted laser desorption/ionization tandem mass spectrometry (MALDI-MS/MS) and matrix-assisted laser desorption/ionization imaging mass spectrometry (MALDI-IMS).

If the clinical method of the invention comprises assaying a biological sample obtained from a subject suspected of having prostate cancer for fPSA^{MGL}, said assaying can be carried out in essentially the same manner as described above for assaying for glycovariants of CA19-9-bearing entities. As is evident to a skilled person, instead of using a CA19-9 binding agent, a fPSA-binding agent such an anti-fPSA antibody, preferably a monoclonal anti-fPSA antibody, more preferably an antigen-binding fragment thereof, even more preferably a site-specifically biotinylated antigen binding fragment thereof, is used in assay formats that are based on binding reactions. The glycan binder is preferably MGL or another lectin or an anti-glycan antibody capable of recognizing the same glycan structure as MGL, Either the fPSA-binding agent or the glycan binder is immobilized of solid surface, the other being detectably labelled directly or indirectly.

However, especially if the biological sample is a blood sample, such as plasma (e.g. EDTA plasma) or serum, a spot assay format should be applied for assaying fPSA^{MGL}. In such embodiments, the capturing agent is preferably an antigen-binding fragment of an anti-fPSA antibody, such as a Fab, F(ab)₂ or F(ab′)₂ fragment immobilized as a dense spot on a small area of a multi-well plate, such as a microtiter well. In some embodiments, the diameter of the spot area is 1-5 mm, 1-3 mm, or 2.5-3 mm and/or the concentration of the capturing agent to be immobilized is from 25 to 150 μg/ml, preferably from 50 to 125 μg/ml or from 75 to 100 μg/ml. In some embodiments, the capturing agent is preferably a site-specifically biotinylated antigen-binding fragment of an anti-fPSA antibody, thereby enabling immobilization thereof in an orderly manner in a desired orientation determined by the selected site of biotinylation. Means and methods for site-specific biotinylation are readily available in the art.

Anti-fPSA antibodies as well as antigen binding fragments and single-chain variants thereof are available from different sources, and further such molecules may be produced according to methods well known in the art.

Moreover, it is also possible to assay a sample for fPSA^{MGL} carried out by NMR, nuclear magnetic resonance (NMR), electrophoresis, chromatographic methods and mass spectrometry, or any appropriate combinations thereof.

### Kits

The present disclosure also provides a kit for use in the present clinical methods and various embodiments thereof. The kit comprises reagents for assaying a biological sample for CA19-9-bearing entities capable of specific binding to at least one lectin selected from the group MBL, WGA and MGL. In some preferred embodiments, the kit comprises reagents for assaying the biological sample for CA19-9-bearing entities capable of specific binding to MBL and, optionally also for CA19-9-bearing entities capable of specific binding to either WGA or MBL. In some further embodiments, the kit may comprise reagents for assaying the biological sample for three different glycovariants of CA19-9-bearing entities, each variant being capable of specific binding to a different lectin selected from MBL, WGA and MGL.

For each glycovariant of CA19-9-bearing entities to be assayed, at least one reagent is a CA19-9-binding agent specific for CA19-9, such as monoclonal anti-CA19-9 antibody, and at least one other reagent is a glycan binder, such as a lectin or an anti-glycan antibody, specific for the glycan structure in question (i.e., specific for the glycan structure capable of specific binding to either MBL, WGA or MGL). For each glycovariant, the same or different CA19-9-binding agents may be employed. Preferably, either the CA19-9-binding agent or the glycan binder is immobilized on a solid surface, such as a microtiter plate. In some further embodiments, streptavidin coating of the solid surface and biotinylation of the binder molecule in question are used for said immobilization. Alternative ways of achieving the same are readily available for a skilled person. If the CA19-9-binding agent is immobilized on the solid surface in order to be used as a capturing agent, the glycan binder is directly or indirectly detectably labelled in order to be used as a tracer. In some embodiments, the glycan binder may be indirectly labelled through a detectable nanoparticle on which it is immobilized. In some other embodiments, the CA19-9-binding agent is used as a tracer, whereas the glycan binder is used as a capturing agent.

For assaying for a CA19-9-bearing entity comprising a glycan structure capable of specific binding to MBL, MBL may be used as the glycan binder. Alternativity, an anti-glycan antibody specific for the same glycan structure may be employed. It is also envisaged that other lectins may be employed as well, provided that they are capable to specifically recognize the same glycan structures as MBL.

Likewise, for assaying for a CA19-9-bearing entity comprising a glycan structure capable of specific binding to WGA, WGA may be used as the glycan binder. Alternativity, an anti-glycan antibody specific for the same glycan structure may be employed. It is also envisaged that other lectins may be employed as well, provided that they are capable to specifically recognize the same glycan structures as WGA.

Likewise, for assaying for a CA19-9-bearing entity comprising a glycan structure capable of specific binding to MGL, MGL may be used as the glycan binder. Alternativity, an anti-glycan antibody specific for the same glycan structure may be employed. It is also envisaged that other lectins may be employed as well, provided that they are capable to specifically recognize the same glycan structures as MGL.

Optionally, the kit may also comprise a control for comparing to an assayed level a glycovariant of a CA19-9-bearing entity in question. In some embodiments, the control is a threshold value for comparing to said assayed level.

In some embodiments, the kit may further comprise reagents for assaying a sample for one or more known prostatic biomarkers such as one or more kallikrein biomarkers including total PSA, free PSA, intact PSA, total hK2 and free hK2. Reagents suitable for such assaying are known to those skilled in the art, and are readily available.

In some embodiments, the kit may alternatively or additionally comprise reagents for assaying a sample for fPSA^{MGL}. Preferably, such reagents include a fPSA-binding agent specific for fPSA, such as an anti-fPSA antibody, preferably a monoclonal anti-fPSA antibody, more preferably an antigen-binding fragment thereof, such as a Fab, F(ab)₂ or F(ab′)₂ fragment, even more preferably a site-specifically biotinylated antigen fragment, such as Fab, F(ab)₂ or F(ab′)₂ fragment, and a glycan binder, such as a lectin or an anti-glycan antibody, specific for a glycan structure comprised on free PSA and capable of specific binding to MGL. In some preferred embodiments, the glycan binder is MGL. In some embodiments, especially in those concerning urine samples or tissue lysates, either the fPSA-binding agent or the glycan binder is used as a capturing agent and is immobilized on a solid surface, the other of the two being used as a tracer and being directly or indirectly detectably labelled. In some other preferred embodiments, especially in those intended for analyzing blood samples, the capturing agent is an antigen-binding fragment of an anti-fPSA antibody, such as a Fab, F(ab)₂ or F(ab′)₂ fragment, preferably a site-specifically biotinylated antigen-binding fragment, such as Fab F(ab)₂ or F(ab′)₂ fragment, immobilized on a solid surface at an area having a diameter smaller than the diameter of the solid support, and the tracer is a glycan binder (e.g. an antibody or lectin) specific for a glycan structure comprised on free PSA and capable of specific binding to MGL, preferably MGL, more preferably a directly or indirectly detectably labelled MGL-nanoparticle. Accordingly, provided herein is a kit comprising i) a multi-well plate, preferably a microtiter plate, immobilized with said capturing agent as disclosed above, and ii) said tracer agent.

In some embodiments, the kit comprises a multi-well plate, such as a microtiter plate, comprising one or more wells immobilized with a CA19-9-binding agent and one or more different wells immobilized with a fPSA-binding agent as disclosed above. The kit also comprises at least two directly or indirectly detectably labelled glycan binders, one specific for a glycan structure capable of specific binding to MBL and one capable of specific binding to MGL. In accordance with what is disclosed above, additional glycan binders may optionally be included in the kit. In some embodiments, the glycan binders may be provided as immobilized on nanoparticles.

In some embodiments, the kit comprises a multi-well plate, such as a microtiter plate, comprising one or more wells having a bottom surface comprising at least two distinct areas or spots, one immobilized with a CA19-9-binding agent and one immobilized with a fPSA-binding agent as disclosed above. The kit also comprises at least two directly or indirectly detectably labelled glycan binders, one specific for a glycan structure capable of specific binding to MBL and one capable of specific binding to MGL. In accordance with what is disclosed above, additional glycan binders may optionally be included in the kit. In some embodiments, the glycan binders may be provided as immobilized on the same or different nanoparticles.

In some further embodiments, the kit may also comprise instructions for performing any method of the present disclosure. In some further embodiments, the kit may also comprise a computer readable medium comprising computer-executable instructions for performing any method of the present disclosure.

Various details and embodiments of the present clinical methods apply also to the present kit, as is readily understood by a skilled person. Thus, properties and features of suitable nanoparticles, for instance, are not repeated herein with respect to the kit.

Also provided is use of the biomarkers and kits disclosed herein for determining prostate cancer disease state in a subject, Accordingly, provided is use of a binder molecule selected from the group consisting of binder molecules specific for a glycan structure capable of specific binding to MBL, binder molecules specific for a glycan structure capable of specific binding to WGA and binder molecules specific for a glycan structure capable of specific binding to MGL for determining the presence or absence of a prostate cancer-associated CA19-9-bearing entity in a sample. Also provided is use of MBL or a binder molecule specific for a glycan structure capable of specific binding to MBL for diagnosing determining prostate cancer disease state in a subject. Any details and specifics disclosed with respect to the present methods and kits, and their various embodiments, apply to the various uses of the biomarkers and kits of the invention even though the details and specifics are not repeated herein.

### Non-Exhaustive List of Numbered Embodiments

Some embodiments of the invention are numbered below.
1. A glycan structure comprised on a carbohydrate antigen 19-9 (CA19-9) bearing molecular entity and capable of specific binding to Mannose binding lectin (MBL) as a biomarker for prostate cancer.
2. A glycan structure comprised on a carbohydrate antigen 19-9 (CA19-9) bearing molecular entity and capable of specific binding to Wheat germ agglutinin (WGA) as a biomarker for prostate cancer.
3. A glycan structure comprised on a carbohydrate antigen 19-9 (CA19-9) bearing molecular entity and capable of specific binding to Macrophage galactose-type lectin (MGL) as a biomarker for prostate cancer.
4. A method of determining prostate cancer disease state in a subject, the method comprising:
a) assaying a sample obtained from said subject for the level of a CA19-9-bearing entity comprising a glycan structure capable of specific binding to MBL, WGA or MGL;
b) comparing the assayed level obtained in step a) with that of a control sample or a predetermined threshold value; and
c) determining the prostate cancer disease state on the basis of said comparison.
5. The method according to embodiment 4, wherein the sample is assayed for the level of at least two different CA19-9-bearing entities comprising glycan structures capable of specific binding to different lectins selected from the group consisting of MBL, WGA and MGL.
6. The method according to embodiments 3 or 4, wherein increased level of said CA19-9-bearing entity in the sample as compared with the level said CA19-9-bearing entity in the control sample or as compared with the predetermined threshold value indicates that said subject has or is at risk of having prostate cancer.
7. The method according to any one of embodiments 2-6, wherein the assaying is carried out by using a binder molecule specific for the glycan structure or by using mass spectrometry, nuclear magnetic resonance (NMR) spectroscopy, electrophoresis, chromatography or a combination thereof.
8. The method according to embodiment 7, wherein the binder molecule is MBL, WGA, MGL or an antibody specific for the glycan structure capable of specific binding to MBL, WGA or MGL, depending on the biomarker in question.
9. The method according to any one of embodiments 2-8, further comprising assaying said sample for one or more biomarkers selected from the group consisting of total prostate specific antigen (PSA), free PSA, free PSA capable of specific binding to MGL (fPSA^{MGL}), intact PSA, total human kallikrein 2 (hK2) and free hK2.
10. A kit for use in determining prostate cancer disease state in a subject, comprising:
a CA19-9-binding agent and at least one binder molecule specific for a glycan structure capable of specific binding to MBL, WGA or MGL, wherein either said CA19-9-binding agent or said binder molecule comprises a detectable label.
11. The kit according to embodiment 10, wherein the binder molecule is selected from the group consisting of MBL, WGA or MGL, or an antibody specific for the glycan structure capable of specific binding to MBL, WGA or MGL.
12. The kit according to embodiment 10 or 11, further comprising reagents for assaying a sample for one or more biomarkers selected from the group consisting of total prostate specific antigen (PSA), free PSA, free PSA capable of specific binding to MGL (fPSA^{MGL}), intact PSA, total human kallikrein 2 (hK2) and free hK2.
13. Use of a kit according to any one of embodiments 10-12 for determining prostate cancer disease state in a subject.
14. Use of a binder molecule selected from the group consisting of binder molecules specific for a glycan structure capable of specific binding to MBL, binder molecules specific for a glycan structure capable of specific binding to WGA and binder molecules specific for a glycan structure capable of specific binding to MGL for determining the presence or absence of a prostate cancer-associated CA19-9-bearing entity in a sample.
15. Use of MBL or a binder molecule specific for a glycan structure capable of specific binding to MBL for determining prostate cancer disease state in a subject.
16. CA19-9 negativity as a biomarker for aggressive prostate cancer.
17. A method of determining prostate cancer disease state in a subject, the method comprising:
a) assaying a blood sample obtained from said subject for the level of free PSA (fPSA) capable of specific binding to MGL using a spot assay, wherein a fPSA-binding agent immobilized on a solid surface at an area having a diameter smaller than the diameter of the solid support is used for capturing fPSA present in said sample, and a detectably labeled binder capable of specific binding to MGL is used for detecting MGL-binding glycovariant of the captured fPSA;
b) comparing the assayed level obtained in step a) with that of a control sample or a predetermined threshold value; and
c) determining the prostate cancer disease state on the basis of said comparison.
18. The method according to embodiment 17, wherein increased level of said free PSA capable of specific binding to MGL in the sample as compared with the level of free PSA capable of specific binding to MGL in the control sample or as compared with the predetermined threshold value indicates that said subject has or is at risk of having prostate cancer.
19. The method according to embodiment 17 or 18, wherein the fPSA-binding agent is an anti-fPSA antibody, preferably an antigen biding fragment thereof, more preferably a Fab fragment, a F(ab)₂ fragment or a F(ab′)₂ fragment, even more preferably a site-specifically biotinylated Fab fragment, F(ab)₂ fragment or F(ab′)₂ fragment.
20. The method according to any one of embodiments 17-19, further comprising assaying said sample for one or more biomarkers selected from the group consisting of total prostate specific antigen (PSA), free PSA, intact PSA, total human kallikrein 2 (hK2) and free hK2.
21. The method according to any one of embodiments 17-20, further comprising assaying said sample for the level of at least one CA19-9-bearing entity comprising a glycan structure capable of specific binding to a lectin selected from the group consisting of MBL, WGA and MGL.
22. A kit for use in determining prostate cancer disease state in a subject, comprising:
i) a multi-well plate with one or more wells having a bottom surface comprising an area immobilized with a fPSA-binding agent, wherein said area has a diameter smaller than the bottom surface; and
ii) a binder specific for a glycan structure capable of specific binding to MGL.
23. The kit according to embodiment 22, wherein the fPSA-binding agent is an anti-fPSA antibody, preferably an antigen biding fragment thereof, more preferably a Fab fragment, a F(ab)₂ fragment or a F(ab′)₂ fragment, even more preferably a site-specifically biotinylated Fab fragment, F(ab)₂ fragment or F(ab′)₂ fragment.
24. The kit according to embodiment 22 or 23, wherein the binder is MGL or an antibody specific for the glycan structure capable of specific binding to MGL.
25. The kit according to any one of embodiments 22-24, wherein the binder is detectably labelled.
26. The kit according to any one of embodiments 22-25, wherein the binder is immobilized on a nanoparticle.
27. The kit according to any one of embodiments 22-26, further comprising reagents for assaying a sample for one or more biomarkers selected from the group consisting of total prostate specific antigen (PSA), free PSA (fPSA), free PSA capable of specific binding to MGL (fPSA^{MGL}), intact PSA, total human kallikrein 2 (hK2) and free hK2.
28. The kit according to any one embodiments 22-27, wherein the multi-well plate is further immobilized with a CA19-9 binding agent, either on one or more different wells than the fPSA-binding agent, or on an area at the bottom of one or more different wells, each area having a diameter smaller than the diameter of the well and each area being distinct from the area immobilized with the fPSA-binding agent; and wherein the kit further comprises a binder specific for a glycan structure capable of specific binding to MBL.
29. The kit according to any one of embodiments 22-28, comprising:
i) a multi-well plate with one or more wells having a bottom surface comprising at least two distinct areas, one immobilized with a CA19-9-binding agent and one immobilized with a fPSA-binding agent; and
ii) at least two binders, one specific for a glycan structure capable of specific binding to MBL and one capable of specific binding to MGL.

### EXAMPLES

### Example 1. CA19-9 Glycovariant Assays

### Anti-CA19-9 Antibodies

The anti-CA19-9 antibody c192 obtained from Fujirebio Diagnostics (Göteborg, Sweden). To yield c192 F(ab′)2 fragments, monoclonal antibody (mAb) c192 was digested by incubating with ID-diluent 1 (50 μl per 1 mg mAb) for two hours in reaction buffer (50 mM Tris-HCl, pH 7.0, 1 mM NaCl, 3 mM EDTA) at +37° C. The digestion was stopped by adding 0.2 M N-ethylmaleimide (NEM) for final concentration of 0.02M NEM. The c192 F(ab)′2 antibody fragments were purified using protein G affinity purification.

For use as a solid-phase capture agent, c192 F(ab′)2 fragment was biotinylated for 4 h at room temperature (RT) with a 40-fold molar excess of biotin isothiocyanate using a standard procedure known in the art. The biotinylated antibodies were purified with NAP-5 and NAP-10 gel-filtration columns (GE Healthcare, Schenectady, NY, USA) by using 50 mmol/L Tris-HCl (pH 7.75), containing 150 mmol/L NaCl and 0.5 g/L NaN₃. The labelled antibodies were stabilized with 1 g/L BSA (Bioreba, Nyon, Switzerland) and stored at +4° C.

### Lectins

Lectin Wheat germ agglutinin (WGA) was purchased from Vector Laboratories (United Kingdom). Mannose binding lectin (MBL) and Macrophage galactose-type lectin (MGL) were purchased from R&D Systems (Minneapolis, Minnesota, United States).

Lectins were immobilized onto europium chelate-doped, monodisperse, carboxyl-modified Fluoro-Max™ polystyrene nanoparticles (97 nm in diameter) which were obtained from Thermo Scientific Seradyn Inc., Indianapolis, IN). The nanoparticles employed produce a long-lifetime fluorescence equivalent to 30,000 chelated ions per particle.

Primary amino groups of lectins were covalently coupled to activated carboxyl groups of the nanoparticles using a procedure described previously with some minor modifications (Soukka et al., Anal. Chem. 2001, 73, 2254-2260). The nanoparticles (1×10¹² particles) were suspended in 10 mmol/L phosphate buffer (pH 7.0), and their surfaces were activated with 0.75 mmol/L N-(3-dimethylaminopropyl)-N-ethylcarbodiimide (Sigma-Aldrich, St. Louis, MO, USA) and 10 mmol/L N-hydroxysulfosuccinimide sodium salt (Sigma-Aldrich). In the coupling reactions, the concentration of WGA was 0.4 mg/ml and MGL and MBL was 0.25 mg/mL, and the reactions contained 100 mmol/L NaCl. The activated particles were mixed with the lectins. The coupling reactions were incubated for 2 h at +23° C. with vigorous shaking. Final washes and blocking of the remaining active groups were performed in Tris-based buffer (10 mmol/L Tris, 0.5 g/L NaN₃, pH 8.5), and the nanoparticle-lectin conjugates were stored in the same buffer supplemented with 2 g/L BSA at 4° C. Before the first instance of use, the particles were mixed thoroughly, sonicated, and centrifuged lightly (350 g, 5 min) to separate non-colloidal aggregates from the monodisperse suspension.

Major carbohydrate binding specificities of the lectins used are shown in Table 1 below.

TABLE 1

| | | Predominant carbohydrate |
|---|---|---|
| Full name | Abbreviation | specificities |
| Mannose binding lectin | MBL | fucose, mannose/mannan |
| Macrophage galactose- | MGL | Terminal α-or β-linked |
| type lectin | | GalNAc |
| Wheat germ agglutinin | WGA | Terminal N-acetylglucosamine |
| | | or chitobiose |

### Other Materials, Reagents and Equipment Used

The 96-well microtiter plates coated with streptavidin (SA plates, product #: 41-07TY), assay wash buffer (product #: 42-01TY) and RED assay buffer (product #: 42-02TY) were from Kaivogen Oy (Turku, Finland). The plate washer (Delfia PlateWash 1296-026) and plate shaker (Delfia PlateWash 1296-026) were from Wallac Oy (Turku, Finland). Time-resolved fluorescence signal from Europium-nanoparticles was measured with Hidex Microplate Reader (Hidex, Finland).

Bromelain solution ID-diluent 1 was from DiaMed (Cressier FR, Switzerland). NAP-5 and NAP-10 buffer exchange columns and Phosphate buffered saline were purchased from GE Healthcare (Chicago Illinois, United States).

### CA19-9 Glycovariant Assays

All the incubations were done in room temperature and in slow shaking. All the assays were performed using yellow streptavidin-coated 96-well microtiter plates.

The CA19-9 standard was prepared for the CA19-9^{MBL} glycovariant assay by using equal amounts of CA19-9 antigen from Fujirebio and Meridian Bioscience and diluted to concentrations of 5, 10, 50, 100 U/mL in TSA-BSA buffer (50 mM Tris-HCl, pH 7.75, 150 mM NaCl, 0.05% NaN₃, 0.5% BSA).

As the standard in CA19-9^{WGA} and CA19-9^{MGL} glycovariant assays CA19-9 antigen from Meridian in concentration of 10, 50, 100, 250 U/mL and from Fujirebio in concentration of 30, 100, 300, 600 U/mL in TSA-BSA buffer (50 mM Tris-HCl, pH 7.75, 150 mM NaCl, 0.05% NaN₃, 0.5% BSA) was used, respectively.

The RED assay buffer (Kaivogen Oy, Turku, Finland) in standard and sample incubation step contained additional 50 mM CaCl₂), 100 mM NaCl for CA19-9^{MBL}, 80 mM CaCl₂ for CA19-9^{WGA} and 50 mM CaCl₂), 150 mM NaCl for CA19-9^{MGL} glycovariant assays.

For immobilization of capturing agents in a whole well assay format, SA plates were washed once and 35 ng, 40 ng or 50 ng of biotinylated anti-CA19-9, bio-c192F(ab′)2, was added in 40 μL volume of RED assay buffer for CA19-9^{MBL}, CA19-9^{WGA} and CA19-9^{MGL} glycovariant assays, respectively. After 1 h incubation the wells were washed twice and 35 μL, 38 μL or 30 μL of assay buffer was added, followed by the addition of 5 μL, 2 μL or 10 μL of clinical plasma samples for CA19-9^{MBL}, CA19-9^{WGA} and CA19-9^{MGL} glycovariant assays, respectively. Calibration curves for each assay type were prepared using 10 μL of each standard dilution disclosed above and 30 μL of assay buffer in triplicates. After 1 h incubation the wells were washed four times and glycan-binding tracer molecules, the lectin-nanoparticles, were added in concentration of 2.5×10⁷, 2.8×10⁷ and 1.2×10⁷ particles per well in 40 μL of RED assay buffer supplemented with 10 mM CaCl₂) for CA19-9^{MBL}, CA19-9^{WGA} and CA19-9^{MGL} assays, respectively. After 90 min incubation the wells were washed six times and the time-resolved fluorescence of Eu was measured with Hidex Microplate Reader.

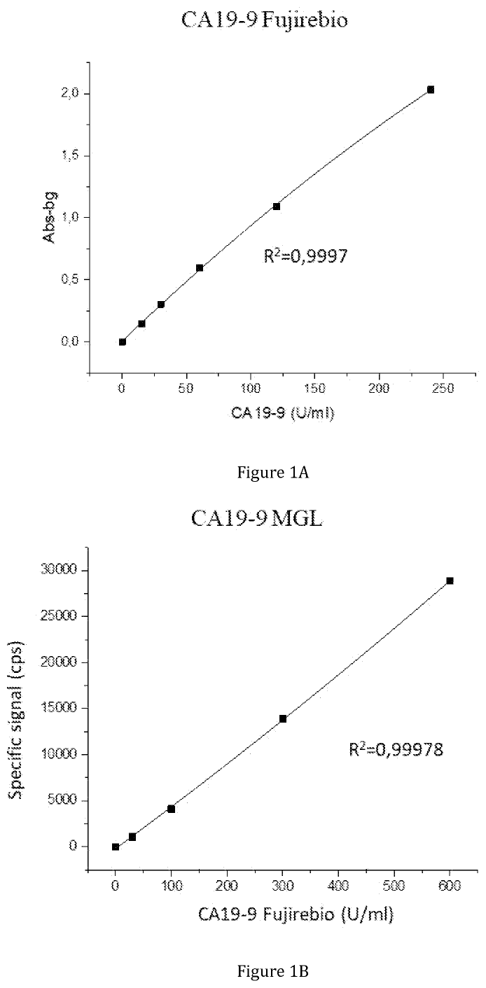

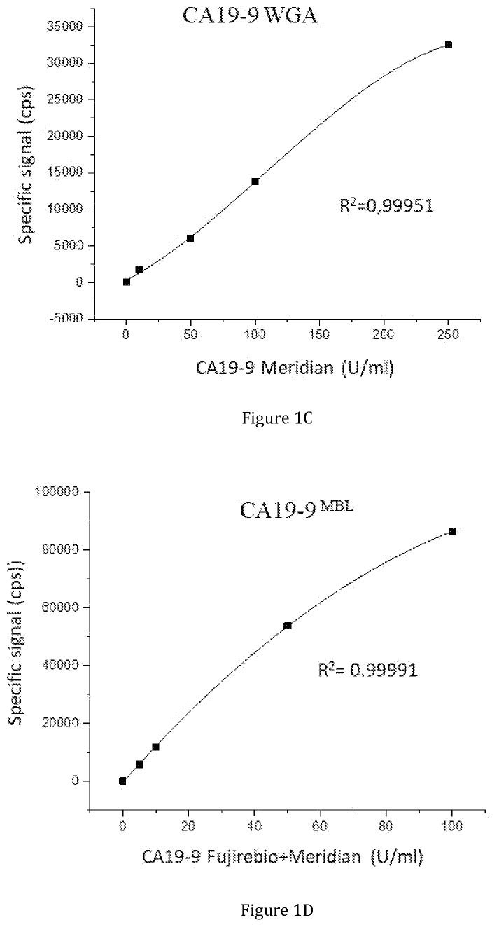

The calibration curves for CA19-9^{MGL} CA19-9^{WGA} and CA19-9^{MBL} glycovariant assays are presented in FIGS. 1B-1D. A calibration curve for a commercial CA19-9 Elisa immunoassay (EIA) (Fujirebio) representing a conventional CA19-9 immunoassay is shown in FIG. 1A.

Notably, the calculated concentrations using the CA19-9 Elisa and the three CA19-glycovariants are not comparable to each other as the glycovariant composition of the calibration standard used is not known.

### Example 2. Analysis of Clinical Samples by the CA19-9 Glycovariant Assays, and Comparison to Conventional CA19-9 Immunoassay

A cohort of clinical samples was analysed side by side with the present CA19-9 glycovariant assays and a conventional CA19-9 immunoassay.

### Clinical Samples

A study cohort of consecutive 249 men with clinical suspicion of prostate cancer (PCa), enrolled in a prospective, registered multi-IMPROD-trial (ClinicalTrials.gov Identifier NCT02241122), was employed. EDTA plasma samples were obtained from Turku, Helsinki, Tampere and Pori University Hospitals with appropriate permissions and informed consent.

For the analyses, the patient's Gleason score (Gle) was used as the “ground truth” and the cohort was dichotomized into three groups, namely into patients with non-cancerous benign condition (n=99), patients with non-significant prostate cancer, i.e., Gleason score 6 graded PCa (n=43) and patients with clinically relevant cancer, i.e., PCa graded as Gleason score 7 or higher (n=107). Table 2 summarizes clinical characteristics for the patient groups.

The age of patients with benign condition or Gleason score 6 graded PCa ranged from 29 to 76 years with a median of 62 years, and the age of the patient with a significant PCa from 40 to 81 years with a median of 67 years.

TABLE 2
Clinical characteristics of patient groups

median (range) or
frequence (percent)

Age at sampling (years)

| | |
|---|---|
| Clinically relevant PCa (n = 107) | 67 (40, 81) |
| Benign or nonsignificant PCa (n = 142) | 62 (29, 76) |

Histological grading

| | |
|---|---|
| Benign | 99 (39.8%) |
| Gleason 6 (3 + 3) | 43 (17.3%) |
| Gleason 7 (3 + 4, 4 + 3) | 74 (29.7%) |
| Gleason 8 (3 + 5, 4 + 4, 5 + 3) | 18 (7.2%) |
| Gleason 9-10 (4 + 5, 5 + 4, 5 + 5) | 15 (6.0%) |

Prostate volume*

| | |
|---|---|
| All | 40 (14, 129) |
| Clinically relevant PCa (n = 107) | 36 (14, 129) |
| Benign or nonsignificant PCa (n = 142) | 41 (14, 106) |

*Volume calculated with ellipsoid formula with MRI

### CA19-9 Glycovariant Assays

The clinical EDTA plasma samples were analysed for CA19-9 glycovariants as described in Example 1. Concentrations of the glycovariants were determined with the aid of calibration curves made as also described in Example 1, and shown in FIGS. 1B-1D.

### Conventional CA19-9 Immunoassay

CA19-9 concentrations in the clinical EDTA plasma samples were analyzed by ELISA using CanAg CA19-9 EIA kit (Fujirebio Diagnostics) according to the manufacturer's instructions with the help of a calibration curve made and shown in FIG. 1A.

### Box-Plot Analyses

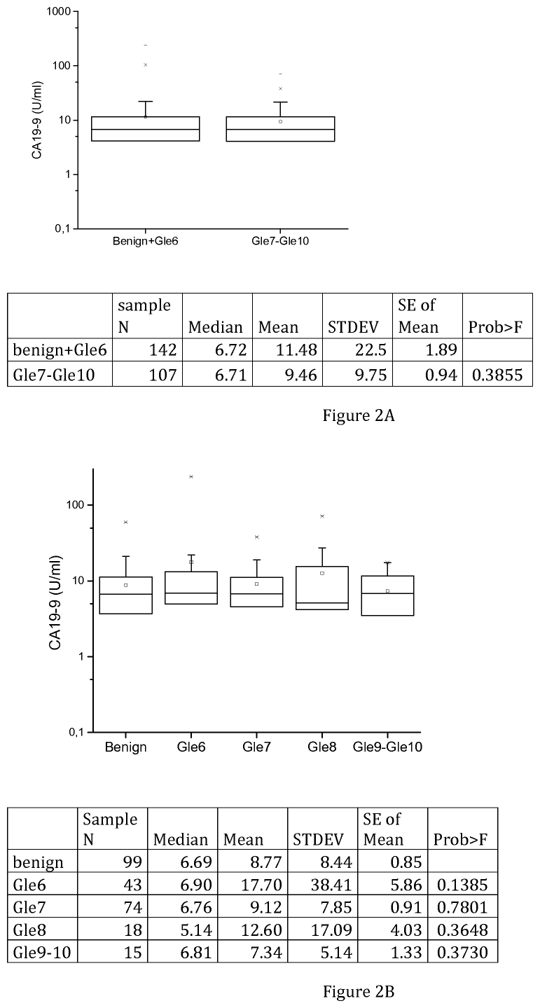

As illustrated in FIGS. 2A and 2B, the conventional CA19-9 ELISA immunoassay provided no separation of clinically relevant cancer groups from the combined group of benign and Gle 6 cancers, nor for the individual cancer groups. Therefore, the conventional CA19-9 assay has no diagnostic value in testing for PCa.

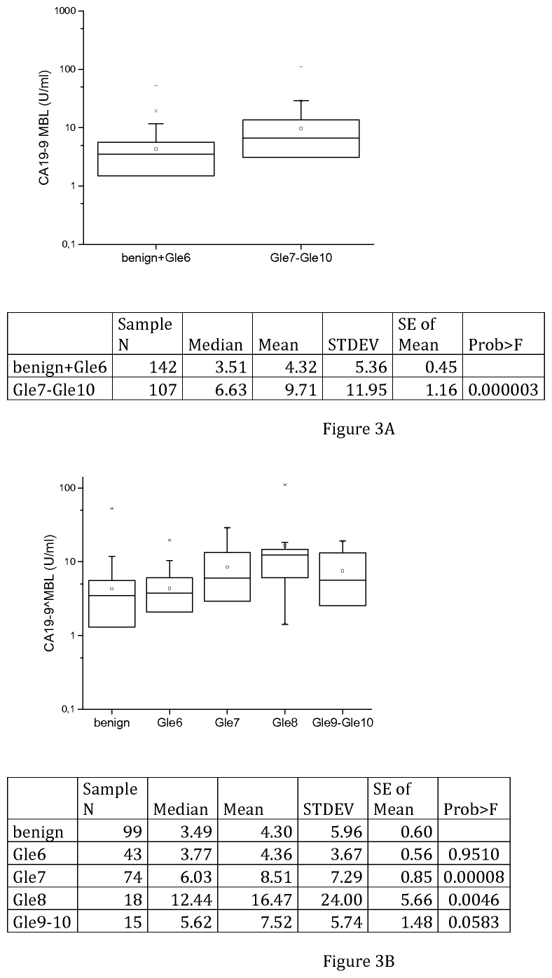

The CA19-9^{MBL} assay in turn provided excellent separation of the medium or high-grade cancer groups from the combined group of benign and Gle 6 cancers, as demonstrated in FIG. 3A. On the other hand, benign cases and Gle 6 cancers could not be separated from each other, as demonstrated in FIG. 3B. To conclude, the CA19-9^{MBL} assay of the invention can be used to distinguish patients with clinically relevant PCa from patients with indolent disease.

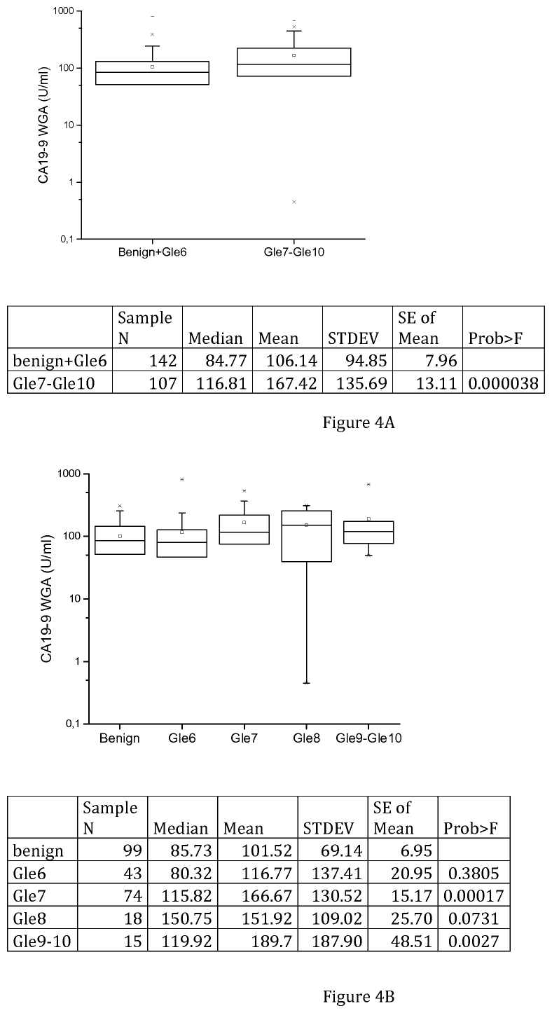

Also the CA19-9^{WGA} assay provided excellent separation of the medium or high-grade cancer groups from the combined group of benign and Gle 6 cancers, as is shown in FIG. 4A. On the other hand, benign cases and Gle 6 cancers could not be separated from each other, as demonstrated in FIG. 4B. To conclude, the CA19-9^{WGA} assay of the invention can be used to distinguish patients with clinically relevant PCa from patients with indolent disease.

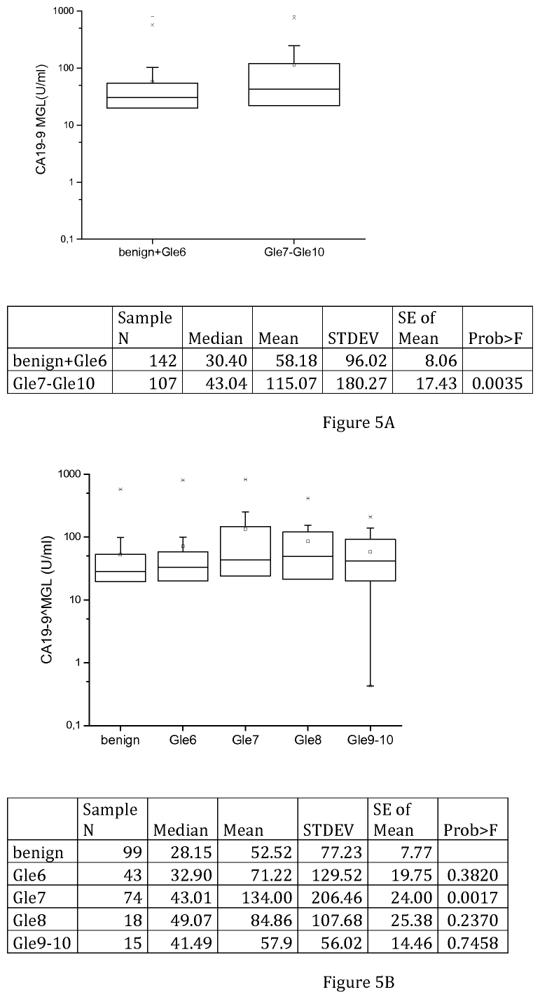

Also the CA19-9^{MGL} assay provided excellent separation of the medium or high-grade cancer groups from the combined group of benign and Gle 6 cancers, as is shown in FIG. 5A. Again, benign cases and Gle 6 cancers could not be separated from each other, as demonstrated in FIG. 5B. To conclude, the CA19-9^{MGL} assay of the invention can be used to distinguish patients with clinically relevant PCa from patients with indolent disease.

### ROC Curves and AUC Values

To evaluate the ability of each variable in detecting GS ≥7, the area under the ROC (receiver operating characteristic) curve (AUC) with the corresponding 95% confidence intervals (CI) were determined.

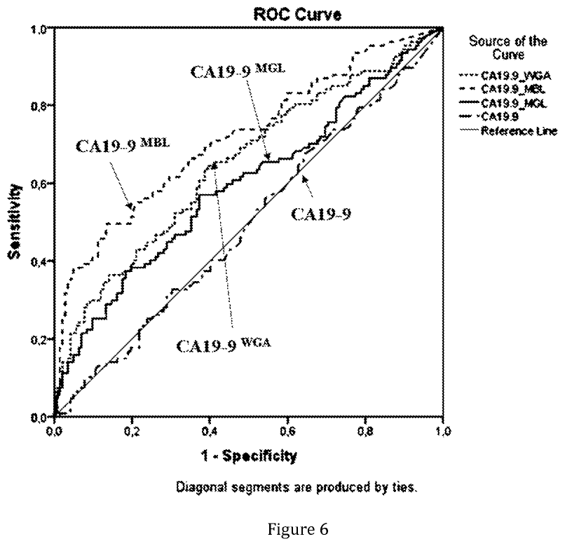

All of the CA19-9 glycovariants separated the clinically relevant cancer groups from the combined group of benign cases and Gleason grade 6 cancers whereas the conventional reference CA19-9 immunoassay did not (AUC 0.495). Based on the AUCs shown in Table 3 below, CA19-9^{MBL} performed the best, followed by CA19-9^{WGA} and CA19-9^{MGL}. CA19-9^{MBL} performed particularly well at specificities of 80-90%. (FIG. 6).

ROC curves were prepared and AUC values determined also for different biomarker combinations. The results demonstrated that the CA19-9 glycovariants complemented each other's diagnostic performance. Further complementation was achieved by assaying the samples also for PSA^{MGL} as described in Example 3 below. The AUC values are shown in Table 3 below.

TABLE 3

| | | | | Asymptotic | |
|---|---|---|---|---|---|
| | | | | 95% | |
| | | | | Confidence | |
| | | | | Interval | |
| | | Std. | Asymptotic | Lower | Upper |
| Test Result Variable(s) | AUC | Error | Sig. | Bound | Bound |
| CA19-9^{MGL} | 0.596 | 0.037 | 0.010 | 0.523 | 0.668 |
| CA19-9^{WGA} | 0.649 | 0.035 | 0.00005 | 0.580 | 0.719 |
| CA19-9^{MBL} | 0.717 | 0.033 | <0.00001*** | 0.652 | 0.783 |
| CA19-9^{MBL} + CA19- | 0.762 | 0.032 | <0.00001*** | 0.699 | 0.824 |
| 9^{WGA} | | | | | |
| CA19-9^{MBL} + CA19- | 0.757 | 0.031 | <0.00001*** | 0.696 | 0.818 |
| 9^{MGL} | | | | | |
| CA19-9^{MGL} + CA19- | 0.657 | 0.035 | 0.00002 | 0.589 | 0.726 |
| 9^{WGA} | | | | | |
| CA19-9^{MBL} + CA19- | 0.782 | 0.030 | <0.00001*** | 0.722 | 0.841 |
| 9^{WGA} + CA19-9^{MGL} | | | | | |
| CA19-9^{MBL} + CA19- | 0.807 | 0.029 | <0.00001*** | 0.751 | 0.863 |
| 9^{WGA} + fPSA^{MGL} | | | | | |
| CA19-9^{MBL} + CA19- | 0.809 | 0.028 | <0.00001*** | 0.753 | 0.864 |
| 9^{WGA} + CA19-9^{MGL} + | | | | | |
| fPSA^{MGL} | | | | | |

### Example 3. Comparison of CA19-9 Glycovariants to Other PCa Markers

Performance of the CA19-9 Glycovariant assays of the invention were compared to the performance of other PCa markers in discriminating between non-significant cases and significant cases from each other.

### Conventional Kallikrein Immunoassays

A multi-kallikrein model, consisting of total PSA (tPSA), free PSA (fPSA), intact PSA (iPSA) and total hK2 (hK2), was used as reference. This multi-kallikrein model is included in the recently FDA-approved 4Kscore test (OPKO Health Inc.) designed to evaluate a patient's likelihood of aggressive PCa. Concentrations of these markers were measured using in-house immunoassays as described previously in Vickers et al. Clin Cancer Res; 16(12), 2010.

### Free PSA^{MGL} Assay

Purified PSA from prostate cancer cell line, LNCaP, and anti-PSA antibody 5A10 Fab were from Department of Biotechnology, University of Turku, Finland. Human MGL was from R&D Systems.

LNCaP PSA was used as a calibrator, and dilutions (1-40 ng/ml LNCaP PSA) were made in Tris-saline-azide buffer (50 mM/L, 150 mmol/L NaCl, 0.5 g/L NaN3, pH 7.75) containing 5 g/L BSA, and used as triplicates.

Biotinylated 5A10 Fab (100 μg/mL) was printed in a spot assay format onto the yellow streptavidin-coated 96-well microtiter plates (Kaivogen Oy, Turku, Finland) with a Nano-Plotter noncontact microdispensing instrument (GeSiM, Germany) by using settings: 70% humidity, pulse 50 s, voltage 90 V, delay 250 s, frequency 100 Hz. Printing buffer contained phosphate buffered saline (pH 7.4) with 10% (v/v) glycerol. The diameter of the of the printed spot was 2.5-3 mm.

Biotinylated 5A10 Fab (100 μg/ml) spotted on to strepavidin-coated microtiter plates was used as a capturing agent in fPSA^{MGL} glycovariant assay. After washing twice, 15 μL of either sample (EDTA plasma) or calibrator and 25 μL of RED Assay buffer with 5 μg/ml native mouse IgG, 5 μg/ml HBR-2, 5 μg/ml MAK-33 was added in triplicates and incubated for 1 hour. MGL-nanoparticles were diluted in RED Assay buffer with 6 mM CaCl₂). After washing four times, the MGL-nanoparticle concentration added was 3×E10⁷ particles per well. The plates were incubated for 90 min and washed six times, followed by measuring of time-resolved fluorescence of Eu.

### Univariate Analysis

To evaluate the capability of each variable (CA19-9^{MBL}, CA19-9^{WGA}, CA19-9^{MGL}, CA19-9, tPSA, fPSA, iPSA, hK2 and fPSA^{MGL}) in detecting Gle ≥7, the area under the ROC (receiver operating characteristic) curve (AUC) with the corresponding 95% confidence intervals (CI) was computed using the DeLong method as known to those skilled the art. From the univariate analysis, all the three different CA19-9 glycovariant assays were able to discriminate the Gle ≥7 cancers from the combined group of benign and Gle 6 cases with high statistical significance; whereas the conventional CA19-9 provided no separation of the two groups. Of four conventional prostatic kallikreins (total PSA, free PSA intact PSA and hK2), total PSA separated the two groups with high statistical significance. Measuring the sub-form of free PSA, namely free PSA glycovariant (fPSA^{MGL}), resulted in the best discrimination (AUC 0.70) of the two groups, which was not possible using the conventional free PSA. (Table 4).

TABLE 4
Median (interquartile range Q1-Q3) and P values of prostatic
kallikreins, PSA glcovariant (PSA MGL), conventional CA19-9
EIA and CA19-9 glycovariants for patients with indolent
disease (Benign + Gleason 6) and patients with clinically
relevant PCa (Gle ≥ 7). AUC with the corresponding
95% (CI) of the markers is also presented for these groups.

| | Benign/Gle = 6 | Gle >= 7 | | |
|---|---|---|---|---|
| | n = 142 | n = 107 | | |
| | Median | Median | U-test | AUC |
| Variable | (Q1-Q3) | (Q1-Q3) | p-value | (95% CI) |
| Total PSA | 9.07 | 11.01 | <.0001** | 0.66 |
| | (6.23-11.91) | (8.6-14.26) | | (0.59-0.73) |
| Free PSA | 1.23 | 1.25 | 0.6249 | 0.52 |
| | (0.77-1.89) | (0.87-1.75) | | (0.45-0.59) |
| Intact | 0.54 | 0.62 | 0.0381* | 0.58 |
| PSA | (0.38-0.75) | (0.43-0.84) | | (0.51-0.65) |
| Total hK2 | 0.10 | 0.13 | 0.0386* | 0.58 |
| | (0.06-0.17) | (0.08-0.18) | | (0.51-0.65) |
| fPSA^{MGL} | 1.20 | 2.56 | <.0001** | 0.70 |
| | (0.65-2.11) | (1.27-3.52) | | (0.63-0.77) |
| CA19-9 | 6.72 | 6.71 | 0.3855 | 0.50 |
| | (4.16-11.52) | (4.09-11.61) | | (0.42-0.57) |
| CA19- | 30.4 | 43.0 | 0.0096* | 0.60 |
| 9^{MGL} | (19.92-54.38) | (22.45-107.5) | | (0.53-0.67) |
| CA19- | 84.75 | 116.8 | <.0001** | 0.65 |
| 9^{WGA} | (51.7-129.75) | (73.8-225.55) | | (0.58-0.72) |
| CA19- | 3.5 | 6.6 | <.0001** | 0.72 |
| 9^{MBL} | (1.52-5.68) | (3.1-13.65) | | (0.65-0.79) |

### Logistic Regression Analyses—Different Combinations

In order to analyze combinations of variables, five logistic regression models were fitted (Model a, b, c, d and e). The results of the analyses are presented in Table 5-9 with odds ratios (OR), 95% CI, and P value. The prediction performance of a model was evaluated by computing the mean AUC and standard deviation (SD) over hold-out cross-validation repeated 10000 times. The hold-out cross-validation consisted in randomly splitting the data into 70% for training the model and 30% for testing.

TABLE 5
Model a: (multi-kallikrein) with corresponding
odd ratios for predicting indolent disease (Benign,
GS = 6) vs clinically relevant PCa (GS >= 7)

MULTI-IMPROD
n = 249

| Variables | OR | 95% CI | P-value |
|---|---|---|---|
| Intercept | 0.1057 | (0.0451-0.2477) | <.0001* |
| totalPSA | 1.2892 | (1.1691-1.4216) | <.0001* |
| freePSA | 0.1878 | (0.0929-0.3795) | <.0001* |
| total_hK2 | 190.9101 | (2.9334-12424.5821) | 0.014* |
| iPSA | 4.6905 | (1.3443-16.3662) | 0.015* |

TABLE 6
Model b: (multi-kallikrein + CA19-9^{MGL} +
CA19-9^{WGA} + CA19-9^{MBL}) with corresponding odd
ratios for predicting indolent disease (Benign, GS = 6)
vs clinically relevant PCa (GS >= 7)

MULTI-IMPROD
n = 249

| Variables | OR | 95% CI | P-value |
|---|---|---|---|
| Intercept | 0.0190 | (0.0060-0.0604) | <.0001** |
| Total PSA | 1.3109 | (1.1771-1.4600) | <.0001** |
| Free PSA | 0.1963 | (0.0928-0.4152) | <.0001** |
| Total hK2 | 27.3834 | (0.2665-2812.8484) | 0.161 |
| Intact PSA | 4.9936 | (1.3665-18.2479) | 0.015* |
| CA19-9^{MGL} | 1.0030 | (1.0004-1.0055) | 0.019* |
| CA19-9^{WGA} | 1.0047 | (1.0018-1.0076) | 0.001* |
| CA19-9^{MBL} | 1.1385 | (1.0708-1.2104) | <.0001** |

In this model, the three best performing parameters were total PSA, free PSA and CA19-9^{MBL} followed by CA19-9^{WGA}. In comparison intact PSA and CA19-9^{MGL} contributed modestly to the model performance.

TABLE 7
Model c (fPSA^{MGL} + CA19-9^{MGL} + CA19-9^{WGA} +
CA19-9^{MBL}) with corresponding odd ratios for predicting
indolent disease (Benign, GS = 6) vs clinically relevant PCa (GS >= 7)

MULTI-IMPROD
n = 249

| Variables | OR | 95% CI | P-value |
|---|---|---|---|
| Intercept | 0.0794 | (0.0390-0.1615) | <.0001** |
| fPSA^{MGL} | 1.4603 | (1.1808-1.8060) | <.0001** |
| CA19-9^{MGL} | 1.0018 | (0.9992-1.0045) | 0.159 |
| CA19-9^{WGA} | 1.0038 | (1.0010-1.0065) | 0.007 |
| CA19-9^{MBL} | 1.1411 | (1.0767-1.2094) | <.0001** |

In this model, the three best contributing parameters were fPSA^{MGL}, CA19-9^{MBL} and CA19-9^{WGA}. The CA19-9^{MGL} did not add to the model.

TABLE 8
Model d (CA19-9^{MGL} + CA19-9^{WGA} + CA19-9^{MBL})
with corresponding odd ratios for predicting indolent
disease (Benign, GS = 6) vs clinically relevant PCa (GS >= 7)

MULTI-IMPROD
n = 249

| Variables | OR | 95% CI | P-value |
|---|---|---|---|
| Intercept | 0.1391 | (0.0766-0.2526) | <.0001** |
| CA19-9^{MGL} | 1.0033 | (1.0010-1.0056) | 0.004* |
| CA19-9^{WGA} | 1.0039 | (1.0012-1.0067) | 0.004* |
| CA19-9^{MBL} | 1.1578 | (1.0921-1.2276) | <.0001** |

In this model, all three CA19-9 glycovariants significantly contributed to the model with CA19-9^{MBL} being the best

TABLE 9
Model e (multi-kallikreins + fPSA^{MGL}) with corresponding
odd ratios (OR). 95% (CI) and p-values for predicting indolent
disease (Benign, GS = 6) vs clinically relevant PCa (GS >= 7)

MULTI-IMPROD
n = 249

| Variables | OR | 95% CI | P-value |
|---|---|---|---|
| Intercept | 0.0518 | (0.0196-0.1367) | <.0001* |
| totalPSA | 1.2784 | (1.1549-1.4150) | <.0001* |
| freePSA | 0.2035 | (0.0974-0.4252) | <.0001* |
| total_hK2 | 96.9659 | (1.2159-7732.9139) | 0.041* |
| iPSA | 4.5965 | (1.0929-19.3307) | 0.037* |
| fPSA^{MGL} | 1.4668 | (1.2000-1.7929) | <.0001* |

In this model, the three best performing parameters were total PSA, free PSA and free PSA^{MGL}. In comparison intact PSA and hK2 contributed modestly to the model performance.

The different models shown here are examples of how biomarkers combinations could be made, where each individual parameter's P-value shows its particular contribution to the model. Final selections of parameters to a model to be used is dependent on a number of considerations, such as availability of biological samples, budget and logistic restrictions for carrying out the laboratory tests, and importantly the type of patient populations included in studies differing with relationship to age, general health status, prostate related clinical conditions etc.

### Model Evaluation Using 10000 Repetitions of Standard Cross-Validation (30% Test 70% Training Data)

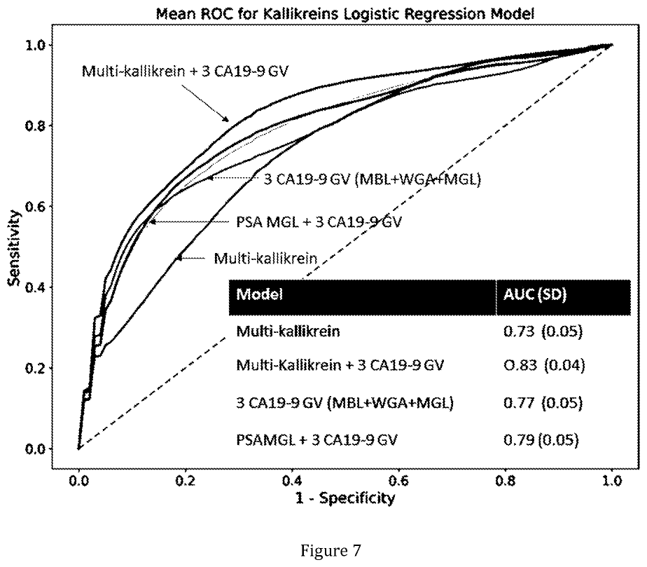

Striking improvements were found with the use of CA19-9 glycovariants. The CA19-9 glycovariants complemented with free PSA glycovariant (PSA^{MGL}) and the four prostatic kallikreins and substantially improved the discriminations, especially at high (80-90)% specificity (FIG. 7, Table 10). Multi-kallikrein model, consisting of total PSA, free PSA, intact PSA and hK2, was used as a reference. The performance was improved when adding the three CA19-9 glycovariants to the multi-kallikrein model, as the AUC increases from 0.73 up to 0.83. The three CA19-9 glycovariants alone showed a better performance over the multi-kallikrein model, as the AUCs for the models were 0.77 and 0.73, respectively. There was further complementation provided by the free PSA^{MGL} glycovariant, as the AUC increased to 0.79 with all four glycovariants.

TABLE 10

| | Average |
|---|---|
| Model | AUC (SD) |
| Multi kallikrein (four parameters) | 0.73 (0.05) |
| Multi kallikrein + CA19-9^{MGL} + CA19-9^{WGA} + | 0.83 (0.04) |
| CA19-9^{MBL} | |
| CA19-9^{MGL} + CA19-9^{WGA} + CA19-9^{MBL} | 0.77 (0.05) |
| fPSA^{MGL} + CA19-9^{MGL} + CA19-9^{WGA} + | 0.79 (0.05) |
| CA19-9^{MBL} | |

### Example 4. Gland Volume Dependency

The dependency of the CA19-9 glycovariants on the prostate gland volume was analyzed. The median prostate gland volume was 40 mL (range 14 to 129 mL) in the whole cohort (n=249). Table 11 shows the results of ROC analyses of Benign/Gle6 vs Gle ≥7 for single parameters in the whole cohort (n=249) and subgroups with gland volume lower or equal (n=133) and higher (n=116) than median (40 mL).

The CA19-9^{MBL} performed equally in the different gland volume groups, whereas CA19-9^{WGA} performed best at prostate gland ≤40 mL group. In comparison total PSA, free PSA, intact PSA and hK2 performed best at ≤median gland volumes and less well with high gland volume group. Unlike the other prostatic kallikreins, the free PSA^{MGL} glycovariant did not show gland volume dependency, performing equally in different gland volume groups. Moreover, CA19-9^{MBL} outperformed all other parameters with no obvious gland volume dependency.

TABLE 11
AUC with the corresponding 95% (CI) and p-value of the single
parameters in whole cohort and different gland volume groups.

| | | | Gland volume ≤ | | Gland volume > | |
|---|---|---|---|---|---|---|
| | All | | median 40 ml | | median 40 ml | |
| | AUC | | AUC | | AUC | |
| | (CI 95%) | p-value | (CI 95%) | p-value | (CI 95%) | p-value |
| CA19-9^{MBL} | 0.717 | <0.000001 | 0.702 | 0.00006 | 0.710 | 0.00018 |
| | (0.652, 0.783) | | (0.613, 0.790) | | (0.607, 0.814) | |
| CA19-9^{WGA} | 0.649 | 0.00005 | 0.703 | 0.00005 | 0.584 | 0.134 |
| | (0.580, 0.719) | | (0.614, 0.792) | | (0.475, 0.693) | |
| CA19-9^{MGL} | 0.596 | 0.0098 | 0.603 | 0.0397 | 0.594 | 0.095 |
| | (0.523, 0.668) | | (0.506, 0.700) | | (0.483, 0.705) | |
| CA19-9 | 0.495 | 0.902 | 0.523 | 0.648 | 0.468 | 0.566 |
| | (0.423, 0.568) | | (0.424, 0.622) | | (0.359, 0.576) | |
| Free PSA^{MGL} | 0.701 | <0.000001 | 0.673 | 0.00058 | 0.704 | 0.00026 |
| | (0.633, 0.770) | | (0.580, 0.766) | | (0.592, 0.816) | |
| Total PSA | 0.662 | 0.00001 | 0.724 | 0.00001 | 0.634 | 0.0166 |
| | (0.595, 0.728) | | (0.639, 0.810) | | (0.528, 0.741) | |
| Free PSA | 0.518 | 0.623 | 0.634 | 0.00786 | 0.490 | 0.859 |
| | (0.446, 0.590) | | (0.539, 0.728) | | (0.379, 0.601) | |
| Intact PSA | 0.577 | 0.03809 | 0.641 | 0.00515 | 0.569 | 0.218 |
| | (0.505, 0.648) | | (0.547, 0.734) | | (0.456, 0.682) | |
| hK2 | 0.577 | 0.03855 | 0.638 | 0.00596 | 0.565 | 0.242 |
| | (0.505, 0.648) | | (0.543, 0.733) | | (0.456, 0.675) | |

Even with lower total AUCs (0.702 and 0.703) the CA19-9^{MBL} and CA19-9^{WGA} glycovariants outperformed the total PSA (AUC 0.724) in low gland volume groups also, as both of the markers had an improved sensitivity at high specificity (80-90%) compared to total PSA.

### Example 5. CA19-9 Negative Cases

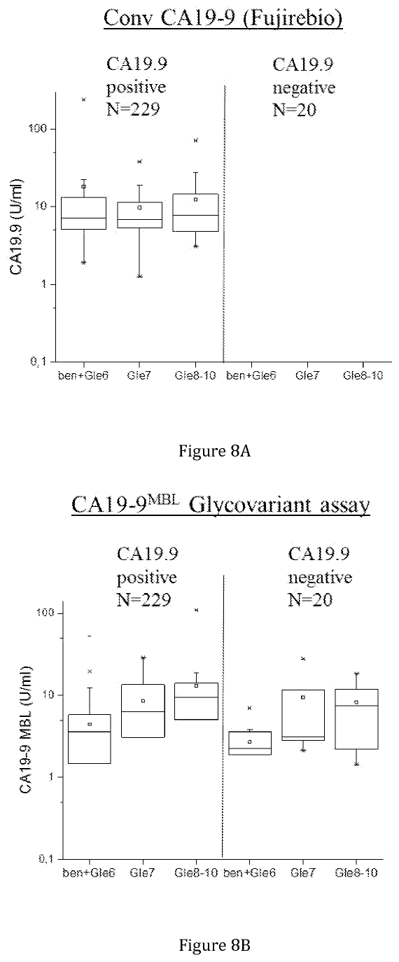

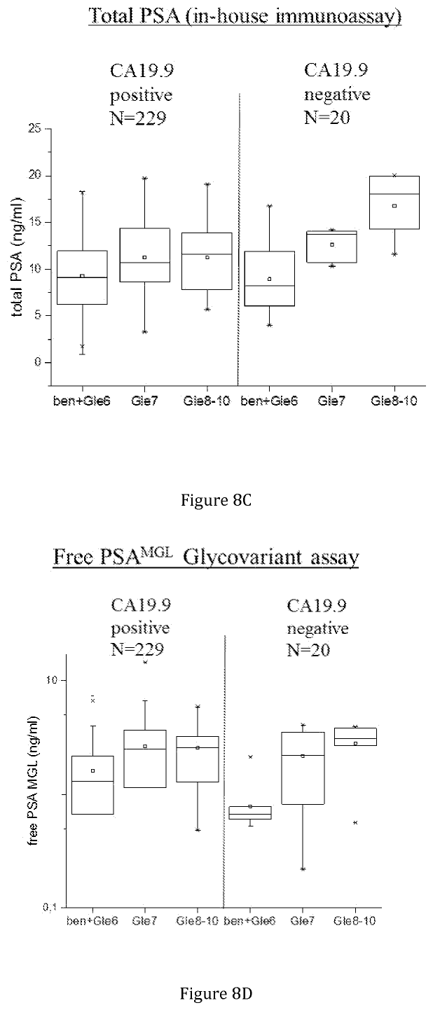

A particular intriguing finding that cancer patients negative for CA19-9 (8% of the cohort) showed signs of a more aggressive disease (as suggested by more pronounced increases of total PSA and free PSA^{MGL} compared to the benign/Gle 6 group). Also the proportion of higher Gleason grades increased in the CA19-9 negative groups. The proportion of Gle8-10 cancers in CA19-9 positive group and negative group was 12% and 25%, respectively. The results are shown in FIGS. 8A-8D.

On the basis of the results, it is envisaged, based on the higher total PSA and free PSA^{MGL} concentrations, that when a patient is Lewis antigen negative, there's tendency towards constituting a more aggressive group.

Accordingly, CA19-9 negativity is suggested as a marker for aggressive PCa.

### Example 6. DC-SIGN Assay (Comparative Example)

Human DC-SIGN (Dendritic Cell-Specific Intercellular adhesion molecule-3-Grabbing Non-integrin) was purchased from R&D Systems. The monoclonal anti-CA19-9 antibody M66106M and CA19-9 antigen were purchased from Meridian Bioscience.

As the standard in CA19-9^{DC-SIGN} glycovariant assay, CA19-9 antigen from Meridian Bioscience was diluted to concentrations of 10, 50, 200, 1000 U/mL in TSA-BSA buffer (50 mM Tris-HCl, pH 7.75, 150 mM NaCl, 0.05% NaN₃, 0.5% BSA).

The RED assay buffer (Kaivogen Oy, Turku, Finland) in standard and sample incubation step contained additional 50 mM CaCl₂) and 90 mM NaCl.

The SA plates were washed once and 60 ng of biotinylated anti-CA19-9 monoclonal antibody, bio-M66106M, was added in 40 μL volume of RED assay buffer. After 1 h incubation the wells were washed twice and 30 μL of assay buffer was added, followed by the addition of 10 μL of either a clinical EDTA plasma sample or a standard dilution. After 1 h incubation the wells were washed four times and glycan-binding reporter molecules, DC-SIGN-nanoparticles (prepared essentially as described for other lectins in Example 1), were added in concentration of 3.0×10⁷ particles per well in 40 μL of RED assay buffer supplemented with 10 mM CaCl₂). After 90 min incubation the wells were washed six times and the time-resolved fluorescence (TRF) of Eu was measured with Hidex. CA19-9^{DC-SIGN} concentrations in the clinical plasma samples were determined with the aid of a calibration curve created on the basis of the measured TRF signals in standard dilutions.

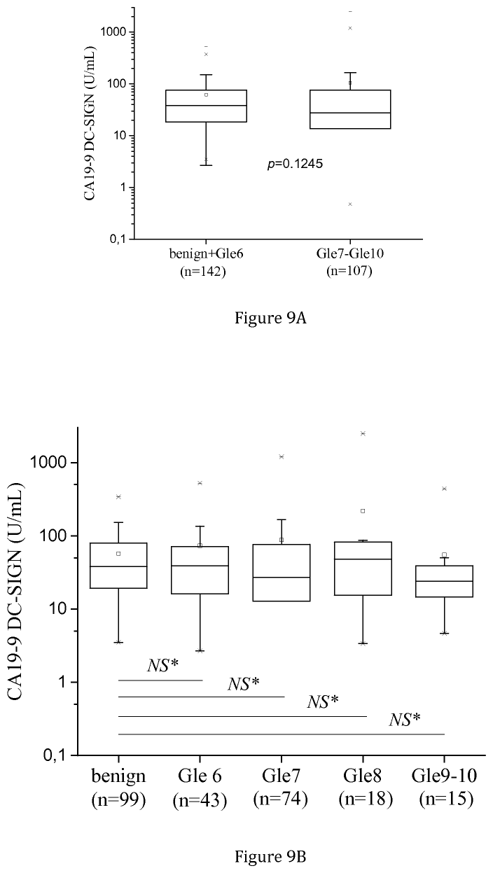

As a result, CA19-9^{DC-SIGN} glycovariant showed no discrimination between clinically relevant PCa and indolent disease (benign and Gle6), nor between benign and different cancer groups (FIG. 9).

### Example 7. Comparison of Free PSA^{MGL} Assay in Spot Assay Format to Whole Well Assay Format

Free PSA^{MGL} assay in a spot assay format was carried out as described in Example 3.

For the conventional whole well glycovariant assay format, 75 ng of biotinylated 5A10 Fab was added into wells in 40 μL volume of RED assay buffer and incubated for an hour in slow shaking. Unbound capture was removed by washing the wells twice and the proceeding protocol followed that of the array-in-well format by adding the calibrator, as described in Example 3.

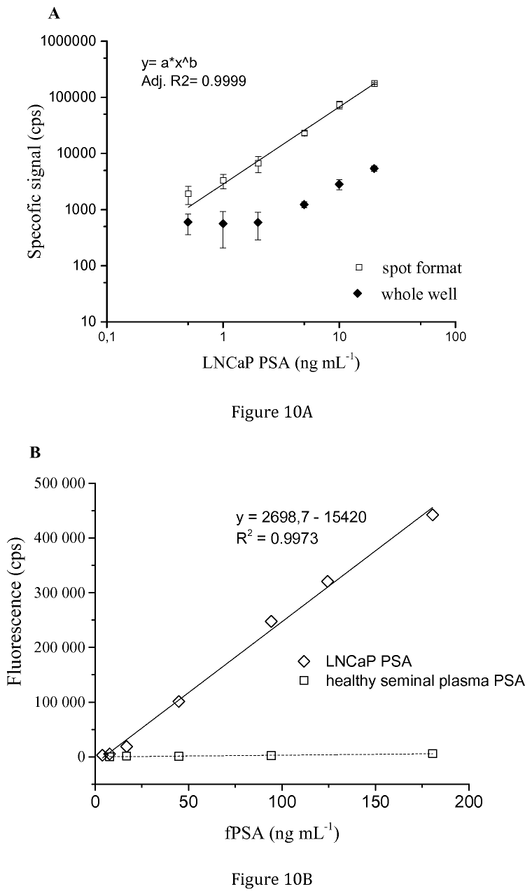

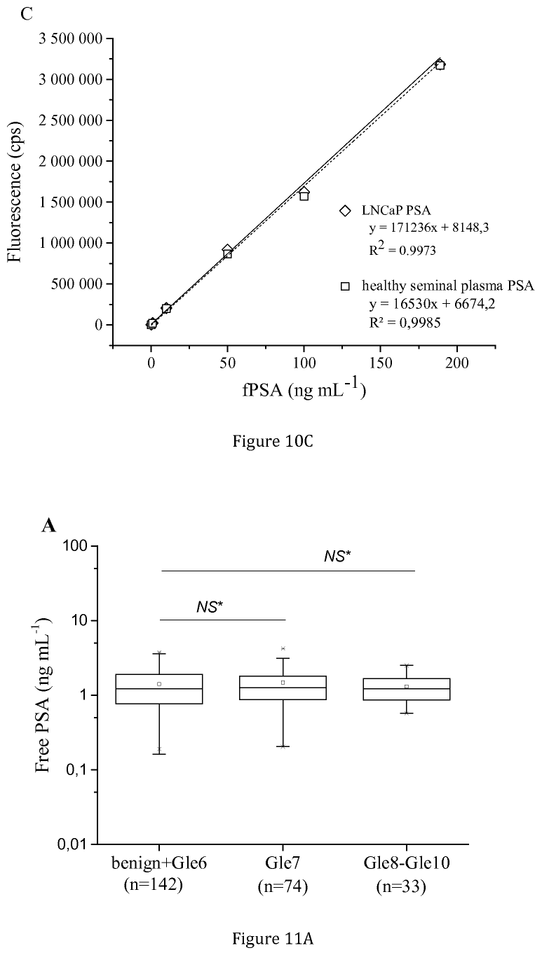

LNCaP PSA was used as a standard in both fPSA^{MGL} assay formats. As shown in FIG. 10A, the signal from the 3 mm spots coated with 5A10 Fab (the spot assay format) were approximately 8-10-fold higher compared to the signal from the conventional whole well assay format, where the free PSA^{MGL} glycovariant was spread over a 15-fold larger area.

The present free PSA^{MGL} glycovariant assay in the spot assay format, was able to distinguish seminal plasma PSA obtained from healthy donors from PSA derived from a prostate cancer cell line, LNCaP. The assay detected cancerous PSA only, as it should (FIG. 10B). In contrast, a conventional free PSA immunoassay did not distinguish non-cancerous seminal plasma-derived PSA standards from cancerous LNCaP-derived PSA (FIG. 10C).

### Example 8. Comparison of Free PSA^{MGL} Assay to Conventional Free PSA Immunoassay

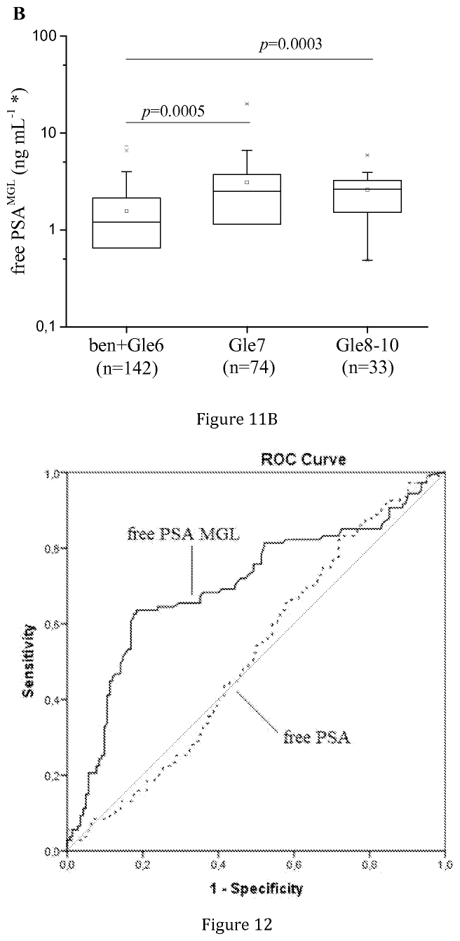

In addition to comparative analyses described in Example 3, box-plot analyses were performed for the present free PSA^{MGL} assay in array-in-well format and for the conventional free PSA immunoassay. According to the results, the free PSA^{MGL} glycovariant assay showed statistically significant discrimination of clinically relevant PCa (Gle ≥7) from benign controls and indolent disease (Gle 6). This applied both for a Gle 7 patient group and to a combined Gle 8-10 patient group (FIG. 11B). Conventional free PSA assay, in turn, was unable to discriminate between patients with a clinically relevant PCa and a benign condition or Gle 6 graded PCa (FIG. 11A). In addition, no significant separation of benign controls and Gle 6 cancers were seen for the free PSA^{MGL} glycovariant, tPSA or fPSA as seen in Table 12 below. The iPSA and hK2 kallikrein forms, in contrast, separated also these two groups.

Although the free PSA^{MGL} glycovariant is a subform of the total free PSA assay, the use of the LNCaP PSA for its calibration creates apparently too high concentrations, very similar to the reference free PSA assay. This is obviously a consequence of lack of knowledge of the relative content of the free PSA^{MGL} glycovariant in the LNCaP PSA preparation.

TABLE 12
Discrimination of indolent (Gle 6) and significant (Gle ≥ 7) PCa's from benign controls
with free PSA^{MGL} glycovariant and the four conventional kallikreins (tPSA, fPSA, iPSA and
hK2). Median values with interquartile range (IQR) are presented and corresponding p-values.

| | | | | |
|---|---|---|---|---|
| fPSA^{MGL} | tPSA | fPSA | iPSA | hK2 |

Benign patients (n = 99)

| | | | | | |
|---|---|---|---|---|---|
| Median ng/mL (IQR) | 1.19 (0.68-2.08) | 9.11 (6.20-11.68) | 1.21 (0.77-1.82) | 0.53 (0.35-0.67) | 0.09 (0.06-0.14) |

Gle 6 PCa patients (n = 43)

| | | | | | |
|---|---|---|---|---|---|
| Median ng/mL (IQR) | 1.21 (0.64-2.18) | 8.44 (6.39-12.21) | 1.24 (0.79-2.08) | 0.62 (0.43-0.82) | 0.15 (0.08-0.19) |
| p-value | 0.762 | 0.342 | 0.423 | 0.045 | 0.013 |

Gle 7-10 PCa patients (n = 107)

| | | | | | |
|---|---|---|---|---|---|
| Median ng/mL (IQR) | 2.56 (1.27-3.53) | 11.01 (8.60-14.26) | 1.25 (0.87-1.75) | 0.62 (0.43-0.84) | 0.13 (0.08-0.18) |
| p-value | 0.00004 | 0.00001 | 0.647 | 0.005 | 0.001 |

Abbreviations:
n = number of patients,
IQR = interquartal range,
Gle = Gleason Score

FIG. 12 shows ROC curves for the free PSA^{MGL} assay and for the conventional free PSA immunoassay, whereas their AUC values with the corresponding 95% confidence intervals (CI) are shown in Table 4, Example 3. The results demonstrate that while fPSA^{MGL} separated the clinically relevant cancer groups from the combined group of benign cases and Gleason grade 6 cancers with an average AUC (95% confidence interval) of 0.70 (0.63, 0.77), the conventional free PSA did not as determined by an average AUC (95% confidence interval) of 0.52 (0.45, 0.59).

### Example 9. PSA^{AAL} Assay (Comparative)

Purified PSA from prostate cancer cell line, LNCaP, and anti-PSA antibody H50 Fab were from Department of Biotechnology, University of Turku, Finland. AAL (Aleuria Aurantia lectin) was purchased from Vector Laboratories (United Kingdom).

LNCaP PSA was used as a calibrator, and dilutions (10-200 ng/ml LNCaP PSA) were made in Tris-saline-azide buffer (50 mM/L, 150 mmol/L NaCl, 0.5 g/L NaN3, pH 7.75) containing 5 g/L BSA, and used as triplicates.

Biotinylated H50 Fab (100 μg/mL) was printed in spot format onto the yellow streptavidin-coated 96-well microtiter plates (Kaivogen Oy, Turku, Finland) with a Nano-Plotter noncontact microdispensing instrument (GeSiM, Germany) as described in Example 3 for the free PSA^{MGL}.

Biotinylated H50 Fab (100 μg/ml) spotted on to streptavidin-coated microtiter plates was used as a capturing agent in PSA^{AAL} glycovariant assay. After washing twice, 15 μL of calibrator or 5 μL or EDTA plasma sample and 25 μL or 35 μL of RED Assay buffer with 5 μg/ml native mouse IgG, 5 μg/ml HBR-2, 5 μg/ml MAK-33 was added in triplicates and incubated for 1 hour. After washing four times, the AAL-nanoparticles, in the concentration of 3.3×10⁷ particles in 40 μL of RED assay buffer supplemented with 8 mM CaCl₂) per well, were added. The plates were incubated for 90 min and washed six times, followed by measuring of time-resolved fluorescence of Eu.

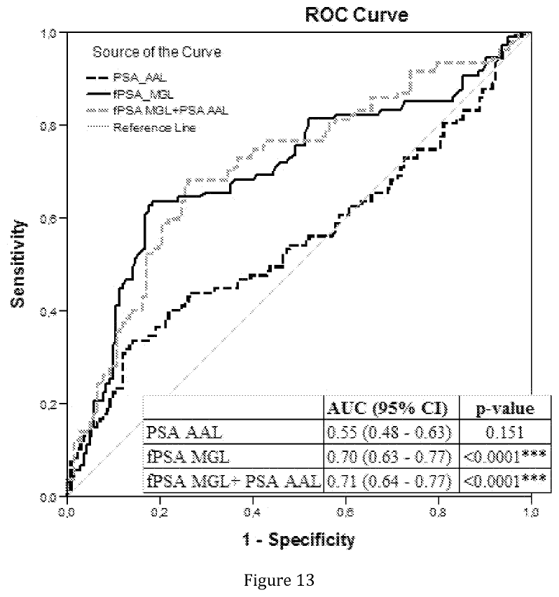

PSA^{AAL} glycovariant was compared to free PSA^{MGL} and the results are shown in FIG. 13. PSA^{AAL} glycovariant showed no discrimination between clinically relevant PCa and indolent disease (benign and Gle6), nor improved the separation when combined with free PSA^{MGL} (FIG. 13).

1. A glycan structure comprised on a carbohydrate antigen 19-9 (CA19-9) bearing molecular entity and capable of specific binding to Mannose binding lectin (MBL) as a biomarker for prostate cancer.

2. A method of determining prostate cancer disease state in a subject, the method comprising:
a) assaying a sample obtained from said subject for the level of a CA19-9-bearing entity comprising a glycan structure capable of specific binding to MBL
b) comparing the assayed level obtained in step a) with that of a control sample or a predetermined threshold value, and
c) determining the prostate cancer disease state on the basis of said comparison.

3. The method according to claim 2, wherein increased level of said CA19-9-bearing entity in the sample as compared with the level said CA19-9-bearing entity in the control sample or as compared with the predetermined threshold value indicates that said subject has or is at risk of having prostate cancer.

4. The method according to claim 3, wherein the assaying is carried out by using a binder molecule specific for the glycan structure or by using mass spectrometry, nuclear magnetic resonance (NMR) spectroscopy, electrophoresis, chromatography or a combination thereof.

5. The method according to claim 4, wherein the binder molecule is Mannose binding lectin (MBL) or an antibody specific for the glycan structure capable of specific binding to MBL.

6. The method according to claim 2, further comprising assaying said sample for a CA19-9-bearing entity capable of specific binding to wheat germ agglutinin (WGA) (CA19-9^{WGA}) and/or for CA19-9 antigens or CA19-9-presenting vesicles capable of specific binding to Macrophage galactose-type lectin (MGL) (CA19-9^{MGL}).

7. The method according to claim 2, further comprising assaying said sample for one or more biomarkers selected from the group consisting of total prostate specific antigen (PSA), free PSA (fPSA), free PSA capable of specific binding to MGL (fPSA^{MGL}), intact PSA, total human kallikrein 2 (hK2) and free hK2.

8. A kit for use in determining prostate cancer disease state in a subject, comprising:
a CA19-9-binding agent and a binder molecule specific for a glycan structure capable of specific binding to MBL, wherein either said CA19-9-binding agent or said binder molecule comprises a detectable label.

9. The kit according to claim 8, wherein the binder molecule is MBL or an antibody specific for the glycan structure capable of specific binding to MBL.

10. The kit according to claim 8, further comprising at least one binder molecule specific for a glycan structure capable of specific binding to WGA and/or a glycan structure capable of specific binding to MGL.

11. The kit according to claim 10, wherein the binder molecule is selected from the group consisting of WGA, MGL, antibodies specific for the glycan structure capable of specific binding to WGA, and antibodies specific for the glycan structure capable of specific binding to MGL.

12. The kit according to claim 8, further comprising reagents for assaying a sample for one or more biomarkers selected from the group consisting of total prostate specific antigen (PSA), free PSA (fPSA), free PSA capable of specific binding to MGL (fPSA^{MGL}), intact PSA, total human kallikrein 2 (hK2) and free hK2.

13. The kit according to claim 8, further comprising:
i) a multi-well plate comprising one or more wells immobilized with a CA19-9-binding agent and one or more different wells immobilized with a fPSA-binding agent; and
ii) at least two binders, one specific for a glycan structure capable of specific binding to MBL and one capable of specific binding to MGL.

14. The kit according to claim 13, wherein said CA19-9-binding agent, said fPSA-binding agent or both are immobilized on an area having a diameter smaller than the diameter of the well.

15. The kit according to claim 8, further comprising:
i) a multi-well plate with one or more wells having a bottom surface comprising at least two distinct areas, one immobilized with a CA19-9-binding agent and one immobilized with a fPSA-binding agent; and
ii) at least two binders, one specific for a glycan structure capable of specific binding to MBL and one capable of specific binding to MGL.

16. The kit according to claim 13, wherein the fPSA binding agent is an anti-fPSA antibody, preferably an antigen binding fragment thereof, more preferably a Fab fragment, a F(ab)₂ fragment or a F(ab′)₂ fragment, even more preferably the fragment being a site-specifically biotinylated fragment.

17.-32. (canceled)
